(19) United States
(12) Patent Application Publication
(10) Pub. No.: US 2026/0061070 A1
(43) Pub. Date: Mar. 5, 2026

(54) COMPOSITIONS AND METHODS FOR TUNABLE MAGNETIC NANOPARTICLES

(71) Applicant: The General Hospital Corporation, Boston, MA (US)

(72) Inventors: Mehmet V. Yigit, Delmar, NY (US); Zdravka Medarova, Methuen, MA (US); Byunghee Yoo, Lexington, MA (US); Eli Mattingly, Lexington, MA (US)

(21) Appl. No.: 19/379,726

(22) Filed: Nov. 4, 2025

Related U.S. Application Data
(63) Continuation of application No. 17/782,369.
(60) Provisional application No. 62/943,927, filed on Dec. 5, 2019.

(51) Int. Cl. A61K 47/69; A61K 9/00; A61K 9/51; A61K 33/26; A61K 47/36; A61K 49/18

(52) U.S. Cl. CPC A61K 47/6929; A61K 9/0009; A61K 9/5115; A61K 9/5161; A61K 9/5192; A61K 33/26; A61K 47/36; A61K 47/6923; A61K 49/1863

(57) ABSTRACT

The present disclosure presents nanoparticle compositions for use in the treatment, prevention, or imaging of a disease (e.g., cancer), methods of treating, preventing, or imaging a disease in a subject in need thereof with the nanoparticle compositions, and methods of preparing the nanoparticle compositions of the disclosure. The nanoparticle compositions can include a magnetic nanoparticle ferric chloride, ferrous chloride, or a combination thereof, and a dextran coating functionalized with one or more amine groups.

### CROSS-REFERENCE TO RELATED APPLICATIONS

This application is a continuation of U.S. patent application Ser. No. 17/782,369, filed on Jun. 3, 2022, which is a U.S. National Phase Application under 35 U.S.C. § 371 of International Patent Application No. PCT/US2020/063635, filed on Dec. 7, 2020, which claims the benefit of U.S. Provisional Application No. 62/943,927, filed on Dec. 5, 2019. The entire contents of the foregoing are incorporated herein by reference in their entireties.

### FEDERALLY SPONSORED RESEARCH OR DEVELOPMENT

This invention was made with government support under CA163461 awarded by the National Institutes of Health. The government has certain rights in the invention.

### TECHNICAL FIELD

The present disclosure presents nanoparticle compositions having tunable magnetic properties and tunable surface modifications (e.g., amine group modifications), methods of preparing these nanoparticle compositions, and methods of using these nanoparticle compositions. The nanoparticle compositions can include ferrous chloride, ferric chloride, dextran, or any combination thereof.

### BACKGROUND

Medical imaging is used to collect information about a subject. In some types of imaging, a contrast agent is administered to the subject. The contrast agent selectively binds to a bioparticle or other structure of interest in the subject. This contrast agent is then detected using a medical imaging device and the collected information is used to develop an image or the like.

Although much information can be gathered from even a single medical image, multiple imaging techniques are necessary to provide comprehensive quantitative diagnostic information having high spatial and temporal resolution, high sensitivity of detection, and tomographic capability. In the past, this has often meant that multiple contrast agents would need to be administered to a single subject for each performed modality.

Multimodal contrast agents have been developed that are suitable for detection by various types of modalities. These multimodal contrast agents typically include multiple entities that are each detectable by a separate modality. The multiple entities are typically joined together using chemical linkers to make particles that each contain all of the respective multiple entities. However, the chemical linkers often have varying stabilities in cells and tissues or across time, meaning that some of the entities could separate, thus degrading the quality and usefulness of these contrast agents.

To avoid the problems of chemically linking multiple entities together, some have attempted to form contrast agents having a core-shell structure. However, to date, there have been significant problems developing a core-shell structure that can be clinically applied. In addition, the currently available particles lack tunable surface functionalization with targeting moieties and tunable magnetic properties.

Hence, a need exists for a multimodal contrast agent that is clinically applicable and provides flexibility of design in terms of surface functionalization and physical properties (e.g. magnetic properties).

### SUMMARY

Certain aspects of the present disclosure are directed to a nanoparticle composition, including: a magnetic nanoparticle including: ferric chloride, ferrous chloride, or a combination thereof, and a dextran coating functionalized with one or more amine groups, wherein the number of the one or more amine groups ranges from about 5 to about 1000.

In some embodiments, the nanoparticle composition includes about 50% weight (wt) to about 100% wt of ferric chloride and about 0% wt to about 50% wt of ferrous chloride. In some embodiments, the nanoparticle composition includes about 0.65 g of ferric chloride and about 0.4 g of ferrous chloride. In some embodiments, the number of the one or more amino groups ranges from about 5 to about 150. In some embodiments, the nanoparticle composition includes about 50% wt to about 100% wt of ferric chloride. In some embodiments, the nanoparticle composition includes about 1.2 g of ferric chloride. In some embodiments, the nanoparticle composition does not comprise ferrous chloride. In some embodiments, the number of the one or more amino groups ranges from about 246 to about 500.

In another aspect, the present disclosure is directed to a nanoparticle composition, including: a magnetic nanoparticle including: ferric chloride, ferrous chloride, or a combination thereof, and a dextran coating, wherein the magnetic nanoparticle has a non-linearity index ranging from about 6 to about 40.

In some embodiments, the nanoparticle composition includes about 50% weight (wt) to about 80% wt of ferric chloride and about 50% wt to about 20% wt of ferrous chloride ferrous chloride. In some embodiments, the nanoparticle composition includes about 0.54 g of ferric chloride and about 0.2 g of ferrous chloride. In some embodiments, the magnetic nanoparticle has a non-linearity index ranging from 8 to 14. In some embodiments, the nanoparticle composition includes about 0% weight (wt) to about 50% wt of ferric chloride and about 100% wt to about 50% wt of ferrous chloride ferrous chloride, or about 80% wt to about [100% wt] of ferric chloride and about 0% wt to about 20% wt of ferrous chloride ferrous chloride. In some embodiments, the nanoparticle composition includes about 0.54 g of ferric chloride and about 0.4 g of ferrous chloride. In some embodiments, the magnetic nanoparticle has a non-linearity index ranging from about 8 to about 67. In some embodiments, the magnetic nanoparticle has a non-linearity index of about 67. In some embodiments, the magnetic nanoparticle has an iron oxide crystal core having a diameter of about 3 nm to about 50 nm, and a hydrodynamic diameter of the magnetic nanoparticle is about 7 nm to about 200 nm.

In some embodiments, the magnetic nanoparticle has a polydispersity of about 0.1 to about 0.25. In some embodiments, the dextran coating includes dextran having a molecular weight ranging from about 1 kDa to about 15 kDa. In some embodiments, the dextran coating includes dextran having a molecular weight of about 10 kDa. In some embodiments, the nanoparticle composition further includes a drug payload attached to a surface of the dextran coating. In some embodiments, the drug payload is an oligonucleotide conjugated to the one or more amine groups. In some embodiments, the drug payload is a drug, an antibody, a growth factor, a nucleic acid, a nucleic acid derivative, a nucleic acid fragments, a protein, a protein derivative, a protein fragment, a saccharide, a polysaccharide fragment, a saccharide derivative, a glycoside, a glycoside fragment, a glycoside derivative, an imaging contrast agent, or any combination thereof.

In another aspect, the present disclosure is directed to a pharmaceutical composition including any nanoparticle composition of the disclosure and at least one pharmaceutically acceptable carrier or diluent.

In another aspect, the present disclosure is directed to a method of imaging a tissue target site in a subject in need thereof, the method including: administering a therapeutically effective amount of any nanoparticle composition of the disclosure to at least the tissue target site at a portion of a body, body part, tissue, cell, or body fluid of the subject; administering energy to the magnetic nanoparticle composition and the tissue target site; detecting a signal of the nanoparticle composition and the tissue target site; and obtaining an image of the tissue target site based on the detected signal.

In some embodiments, the imaging is magnetic resonance imaging, magnetic particle imaging, or a combination thereof, and the energy is a magnetic field. In some embodiments, the disease is cancer, and the tissue target site is a tumor. In some embodiments, the nanoparticle composition accumulates at the target site of the subject.

In another aspect, the present disclosure is directed to any composition of the disclosure for use in a method of imaging a disease in a subject in need thereof.

In another aspect, the present disclosure is directed to a method of preparing any nanoparticle composition of the disclosure, the method including: dissolving dextran in water; crosslinking the dextran with epichlorohydrin; preparing a ferrous chloride solution, a ferric chloride solution, or a combination thereof, preparing a mixture by adding the ferrous chloride solution, the ferric chloride solution, or the combination thereof to the dextran; adding a base to the mixture while stirring and subjecting the mixture to an ice bath; and subjecting the mixture to a temperature of about 75° C. to about 90° C., wherein the step of adding the base prevents the formation of iron oxide crystals, iron oxide hydrates, or a combination thereof, and wherein the mixture includes about 50% weight (wt) to 100% wt of ferric chloride and about 0% wt to 50% wt of ferrous chloride.

In another aspect, the present disclosure is directed to a method of preparing any nanoparticle composition of the disclosure, including: dissolving dextran in water; crosslinking the dextran with epichlorohydrin; preparing a ferrous chloride solution, a ferric chloride solution, or a combination thereof; preparing a mixture by adding the ferrous chloride solution, the ferric chloride solution, or the combination thereof to the dextran; adding a base to the mixture while stirring and subjecting the mixture to an ice bath; and subjecting the mixture to a temperature of about 75° C. to about 90° C., wherein the step of adding the base prevents the formation of iron oxide crystals, iron oxide hydrates, or a combination thereof, and wherein the mixture includes 50% wt to about 80% wt of ferric chloride and about 50% wt about 20% wt of ferrous chloride.

The term “magnetic” is used to describe a composition that is responsive to a magnetic field. Non-limiting examples of magnetic compositions (e.g., any of the nanoparticle compositions described herein) can contain a material that is paramagnetic, superparamagnetic, ferromagnetic, or diamagnetic. Non-limiting examples of magnetic compositions contain a metal oxide selected from the group of: magnetite; ferrites (e.g., ferrites of manganese, cobalt, and nickel); Fe(II) oxides; and hematite, and metal alloys thereof. Additional magnetic materials are described herein and are known in the art.

The term “diamagnetic” is used to describe a composition that has a relative magnetic permeability that is less than or equal to 1 and that is repelled by a magnetic field.

The term “paramagnetic” is used to describe a composition that develops a magnetic moment only in the presence of an externally applied magnetic field.

The term “ferromagnetic” or “ferromagnetic” is used to describe a composition that is strongly susceptible to magnetic fields and is capable of retaining magnetic properties (a magnetic moment) after an externally applied magnetic field has been removed.

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein.

By the term “magnetic nanoparticle” is meant a nanoparticle (e.g., any of the nanoparticles described herein) that is magnetic (as defined herein). Non-limiting examples of magnetic nanoparticles are described herein. Additional magnetic nanoparticles are known in the art.

By the term “nucleic acid” is meant any single- or double-stranded polynucleotide (e.g., DNA or RNA, cDNA, semi-synthetic, or synthetic origin). The term nucleic acid includes oligonucleotides containing at least one modified nucleotide (e.g., containing a modification in the base and/or a modification in the sugar) and/or a modification in the phosphodiester bond linking two nucleotides. In some embodiments, the nucleic acid can contain at least one locked nucleotide (LNA). Non-limiting examples of nucleic acids are described herein. Additional examples of nucleic acids are known in the art.

By the term “imaging” is meant the visualization of at least one tissue of a subject using a biophysical technique (e.g., electromagnetic energy absorption and/or emission). Non-limiting embodiments of imaging include magnetic resonance imaging (MRI), X-ray computed tomography, and optical imaging.

The terms “subject” or “patient,” as used herein, refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used in the specification and the appended claims, the singular forms “a,” “an” and “the” include plural referents unless the context clearly dictates otherwise. Thus, for example, reference to “a nanoparticle” includes mixtures of nanoparticles, reference to “a nanoparticle” includes mixtures of two or more such nanoparticles, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Certain embodiments of the present disclosure include methods of using any of the nanoparticle compositions for the treatment, prevention, diagnosing, and/or imaging of a disease in a subject in need thereof. There is currently a need for tunable and improved nanoparticle compositions that can meet the necessary requirements to successfully reach target sites in the human body for treatment and/or imaging purposes. The nanoparticle compositions and methods of using the nanoparticle compositions of the present disclosure address the above-mentioned necessary requirements. In some embodiments, key physical characteristics (e.g., amination and magnetic strength) of the nanoparticle compositions can be fine-tuned by modulating the concentration of certain components (e.g., concentrations of ferrous chloride or ferric chloride). In some embodiments, the nanoparticle compositions can be scaled-up with no change in physical characteristics (e.g., amination, magnetic strength, size, and polydispersity). In some embodiments, the nanoparticle compositions can have long-term stability (e.g., at least up to 6 months). In some embodiments, the magnetic nanoparticles can be prepared by a precipitation method in aqueous media, which is eco-friendly and cheaper than other synthetic methods.

In some embodiments, the methods of using the nanoparticle compositions described herein can prevent, treat, reduce and/or eliminate symptoms associated with diseases (e.g., cancer). In some embodiments, the methods of using the nanoparticle compositions described herein can aid in the imaging of a target site (e.g., a tumor). In some embodiments, the nanoparticle compositions can be used to simultaneously image and treat a target site (e.g., a tumor) in a subject in need thereof.

In some embodiments, the nanoparticle compositions enable sustained delivery of a payload (e.g. oligonucleotides) to a target site (e.g. a tumor). In some embodiments, the nanoparticle compositions are amenable to delivery of a payload (e.g. oligonucleotides) to target sites that are conventionally difficult to reach for a drug delivery vehicle (e.g., a tumor or tumor core). In some embodiments, the nanoparticle compositions are biocompatible and can remain in blood circulation with a half-life of about 0.25 hours to about 24 hours.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features and advantages of the invention will be apparent from the following detailed description and figures, and from the claims.

### DESCRIPTION OF DRAWINGS

FIG. 1 shows an example of the set-up for dextran dissolution during the method of preparing the nanoparticles of the disclosure.

FIG. 2 shows an example of the set-up for dextran dissolution during the method of preparing the nanoparticles of the disclosure.

FIG. 3 shows an absorbance spectrum of aminated, dextran-coated nanoparticles exposed to 6N-hydrochloric acid and this solution was monitored as measured by ultraviolet/visible light (UV/Vis) spectrometry.

FIG. 4 shows size characterization of “Condition 1” nanoparticles having about 60-90 amine groups per magnetic nanoparticle (MNP); the size was about 11.48 nanometers (nm), as measured by dynamic light scattering.

FIG. 5 shows size characterization of “Condition 2” nanoparticles having about 250 amine groups per MNP; the size was about 15.6 nm, as measured by dynamic light scattering.

FIG. 6 shows an absorbance spectrum of “Condition 2” nanoparticles having about 250 amine groups per magnetic nanoparticle (MNP) as measured by UV/Vis spectrometry.

FIG. 7 shows an absorbance spectrum of “Condition 1” nanoparticles having about 60-90 amine groups per magnetic nanoparticle (MNP) and “Condition 2” nanoparticles having about 246-500 amine groups per MNP, as measured by UV/Vis spectrometry.

FIG. 8 shows an example of gel electrophoresis for the analysis of oligonucleotide loading in Condition 1 MNP. By varying the ratio of oligonucleotides (oligo) to amino groups per nanoparticle, the number of oligos/magnetic nanoparticle (Oligo/MN) can be progressively increased. Oligo/MN numbers represent the molar ratio of oligos per nanoparticle. The number of oligo was tested with 64 amine/MNP, and the reaction ratio varied to maximize the loading of oligo. These MNP were synthesized by the condition in Table 3 (i.e., MNP having an Fe3+/Fe2+ ratio of 1:1) and in the presence of excess ammonium hydroxide addition.

FIG. 9 shows magnetic particle spectrometry for the quantification of magnetic properties of nanoparticles. As a main criterion of magnetic property, non-linearity index was compared between samples having the formulations shown.

FIG. 10 shows example nanoparticles with a 1:1 ratio of Fe³⁺:Fe²⁺ and a non-linearity index of 12.1 having an average nanoparticle size of about 149.3 nm and a standard deviation of 0.9 nm, as measured by dynamic light scattering.

FIG. 11 shows magnetic particle spectrometry for the quantification of magnetic properties of nanoparticles. The non-linearity index of nanoparticles synthesized according to “Condition B” shown in Table 4 was calculated to be 9.7111.

FIG. 12 shows the nanoparticles of FIG. 11 having an average nanoparticle size of about 127.1 nm and a standard deviation of 0.21 nm, as measured by dynamic light scattering.

FIG. 13 shows magnetic particle spectrometry for the quantification of magnetic properties of nanoparticles. The non-linearity index of nanoparticles synthesized according to “Condition C” shown in Table 4 was calculated to be 8.8326. This measurement was taken 1 month after synthesis to check for stability of the nanoparticles.

FIG. 15 shows the nanoparticles of FIG. 14 having an average nanoparticle size of about 63.47 nm and a standard deviation of 0.61 nm, as measured by dynamic light scattering.

FIG. 16 shows magnetic particle spectrometry for the quantification of magnetic properties of nanoparticles. The non-linearity index of nanoparticles synthesized according to “Condition E” shown in Table 4 was calculated to be 14.3731.

FIG. 17 shows magnetic particle spectrometry for the quantification of magnetic properties of nanoparticles. The non-linearity index of nanoparticles synthesized according to “Condition E” shown in Table 4 was calculated to be 15.6437 after being in storage for about 1 month to check for stability of the nanoparticles.

FIG. 18 shows the nanoparticles of FIG. 16 having an average nanoparticle size of about 181.83 nm and a standard deviation of 1.0 nm, as measured by dynamic light scattering, after being in storage for about 2 months.

FIG. 19 shows magnetic particle spectrometry for the quantification of magnetic properties of nanoparticles. The non-linearity index of nanoparticles synthesized according to “Condition F” shown in Table 4 was calculated to be 14.806.

FIG. 20 shows magnetic particle spectrometry for the quantification of magnetic properties of nanoparticles. The non-linearity index of the nanoparticles of FIG. 19 was calculated to be 14.2168 after being in storage for about 1 month.

FIG. 21 shows the nanoparticles of FIG. 19 having an average nanoparticle size of about 185.97 nm and a standard deviation of 0.25 nm, as measured by dynamic light scattering, after being in storage for about 2 months.

FIG. 22 is a schematic illustrating surface modification of example nanoparticles with amine groups for suspension stabilization and surface modification of example nanoparticles with polyethylene glycol-2000 (PEG-2000) for enhanced blood circulation.

### DETAILED DESCRIPTION

The magnetic nanoparticles described herein were discovered to be amenable to having tunable magnetic properties and surface functionalization. Magnetic nanoparticles having these features are provided herein as well as methods of preparing these magnetic nanoparticles and methods of treating, preventing, and/or imaging a disease in a subject in need thereof by administering these magnetic nanoparticles.

### Nanoparticle Compositions

Provided herein are nanoparticles compositions including magnetic nanoparticles including ferric chloride, ferrous chloride, or a combination thereof, and a dextran coating. In some embodiments, the compositions can contain a mixture of two or more of the different nanoparticle compositions described herein. In some embodiments, the compositions contain at least one magnetic nanoparticle having a tunable surface functionalization, and at least one magnetic nanoparticle having tunable magnetic properties.

### Tunable Amine Group Functionalization

In some embodiments, the magnetic nanoparticles can be functionalized with one or more amine groups. In some embodiments, the functionalization occurs at the surface of the magnetic nanoparticles. In some embodiments, the one or more amine groups are covalently linked to the dextran coating. In some embodiments, the one or more amine groups substitute one or more hydroxyl groups of the dextran coating. In some embodiments, the number of the one or more amine groups is tunable based on a concentration of ferric chloride, ferrous chloride, or a combination thereof. In some embodiments, the nanoparticle composition includes about 5 to about 1000 amine groups. In some embodiments, the nanoparticle composition includes about 5 to 25, 25 to 100, 100 to 150, 150 to 200, 200 to 250, 250 to 300, 300 to 350, 350 to 400, 450 to 500, 500 to 550, 550 to 600, 600 to 650, 650 to 700, 700 to 750, 750 to 800, 800 to 850, 850 to 900, 900 to 950, or 950 to 1000 amine groups.

In some embodiments, the magnetic nanoparticles can contain a core of a magnetic material (e.g., ferric chloride and/or ferrous chloride). In some embodiments, the nanoparticle compositions include about 0.60 g to about 0.70 g of ferric chloride and about 0.3 g to about 0.5 g of ferrous chloride. In some embodiments, the nanoparticle compositions including about 0.60 g to about 0.70 g of ferric chloride and about 0.3 g to about 0.5 g of ferrous chloride are functionalized with about 5 to 150 amine groups. In some embodiments, the nanoparticle compositions including about 0.65 g of ferric chloride and about 0.4 g of ferrous chloride are functionalized with about 60 to 90 amine groups. In some embodiments, the nanoparticle compositions including about 0.65 g of ferric chloride and about 0.4 g of ferrous chloride are functionalized with about 5 to 150 amine groups. In some embodiments, the nanoparticle compositions including about 0.65 g of ferric chloride and about 0.4 g of ferrous chloride are functionalized with about 1 to 150 amine groups. In some embodiments, the nanoparticle compositions including about 0.65 g of ferric chloride and about 0.4 g of ferrous chloride are functionalized with about at least 1 to 10 amine groups, 10 to 20 amine groups, about 20 to 30 amine groups, about 30 to 40 amine groups, about 40 to 50 amine groups, about 50 to 60 amine groups, about 60 to 70 amine groups, about 70 to 80 amine groups, about 80 to 90 amine groups, about 90 to 100 amine groups, about 100 to 110 amine groups, about 110 to 120 amine groups, about 120 to 130 amine groups, about 130 to 140 amine groups, or about 140 to 150 amine groups.

In some embodiments, the nanoparticle compositions including about 1 g to about 1.4 g of ferric chloride. In some embodiments, the nanoparticle compositions including about 1 g to about 1.4 g of ferric chloride are functionalized with about 246 to 500 amine groups. In some embodiments, the nanoparticle compositions including about 1.2 g of ferric chloride are functionalized with about 246 to 500 amine groups. In some embodiments, the nanoparticle compositions functionalized with about 246 to 500 amine groups do not include ferric chloride. In some embodiments, the nanoparticle compositions including about 1.2 g of ferric chloride are functionalized with about 200 to 600 amine groups. In some embodiments, the nanoparticle compositions including about 1.2 g of ferric chloride are functionalized with about at least 200 to 250 amine groups, 250 to 300 amine groups, about 300 to 350 amine groups, about 350 to 400 amine groups, about 400 to 450 amine groups, about 450 to 500 amine groups, about 500 to 550 amine groups, about 550 to 600 amine groups, or more.

Thus, in some embodiments, the number of amine groups conjugated to the dextran coating can be fine-tuned by controlling the concentrations of ferric chloride and ferrous chloride, which are used to prepare the magnetic nanoparticles.

### Tunable Magnetic Properties

In some embodiments, the nanoparticle compositions include magnetic nanoparticles having a magnetic strength that is tunable based on a concentration of ferric chloride, ferrous chloride, or a combination thereof.

In some embodiments, the nanoparticle compositions include about 0.1% to about 99.9% of ferric ion and about 99.9% to about 0.1% of ferrous ion in total iron per MNP. In some embodiments, the nanoparticle compositions including about 60% to about 80% of ferric chloride and about 20% to about 40% of ferrous chloride have stronger magnetic properties than nanoparticle compositions having a ferrous chloride amount higher than about 80%. In some embodiments, the nanoparticle compositions including about 70% of ferric ion and about 30% g of ferrous ion have stronger magnetic properties than nanoparticle compositions having a ferrous ion amount higher than about 30%.

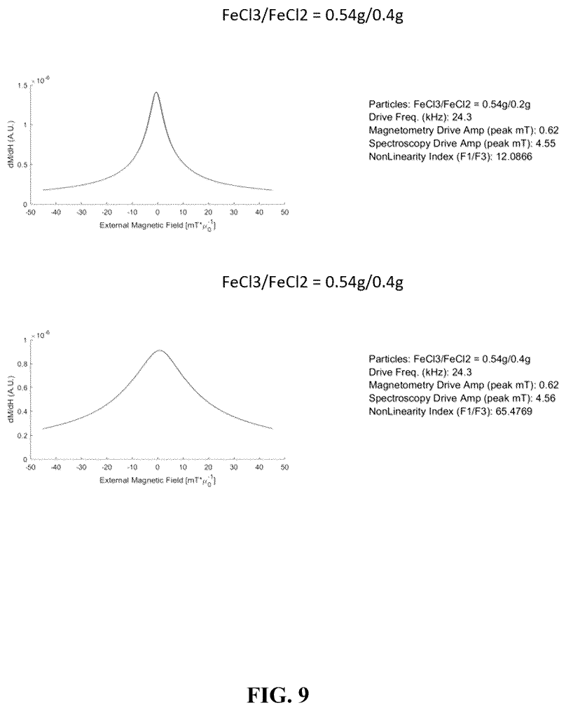

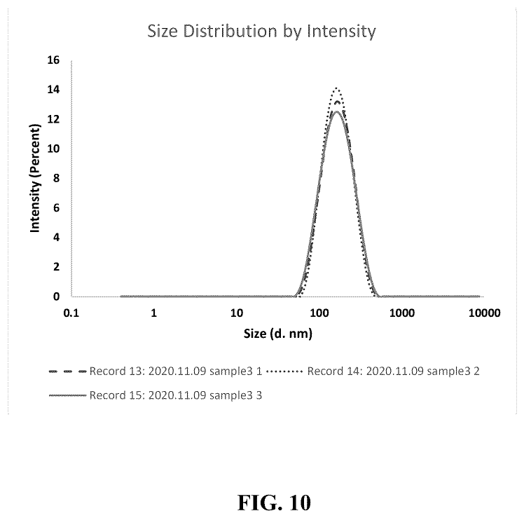

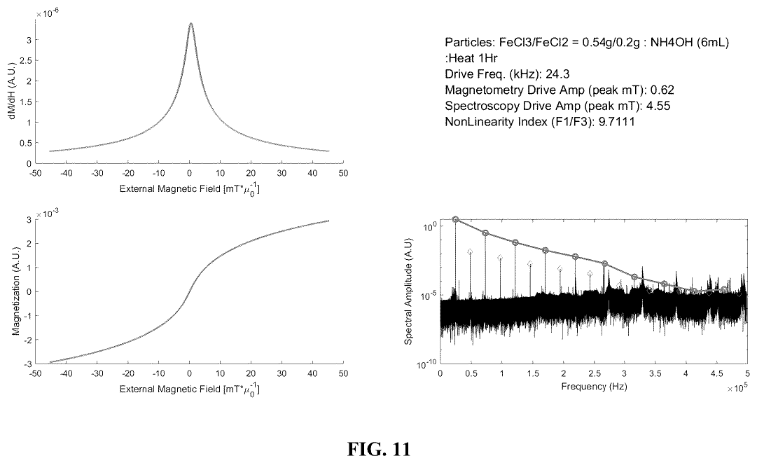

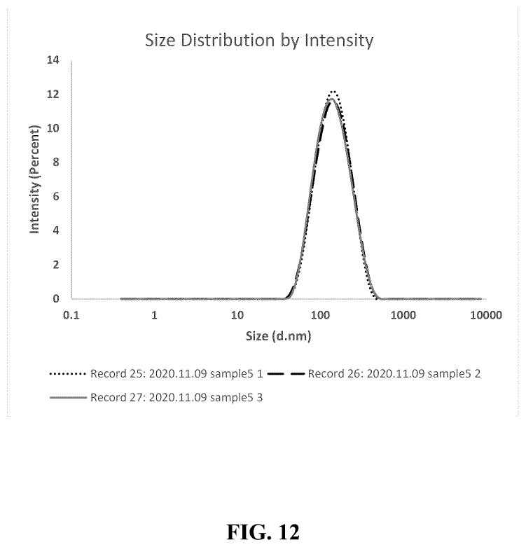

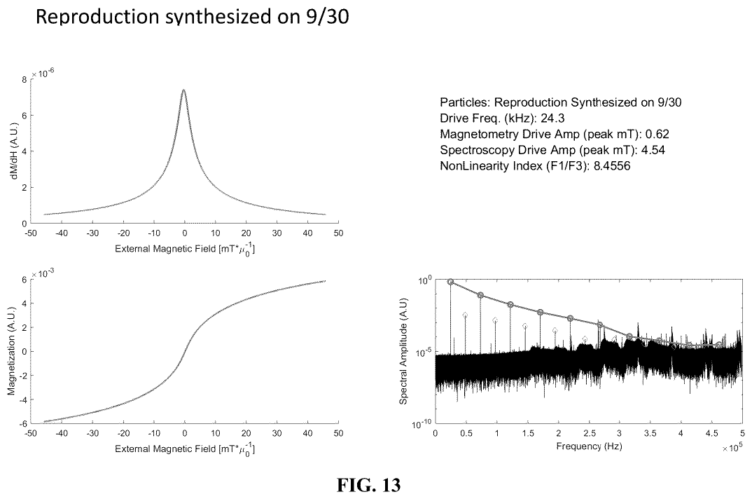

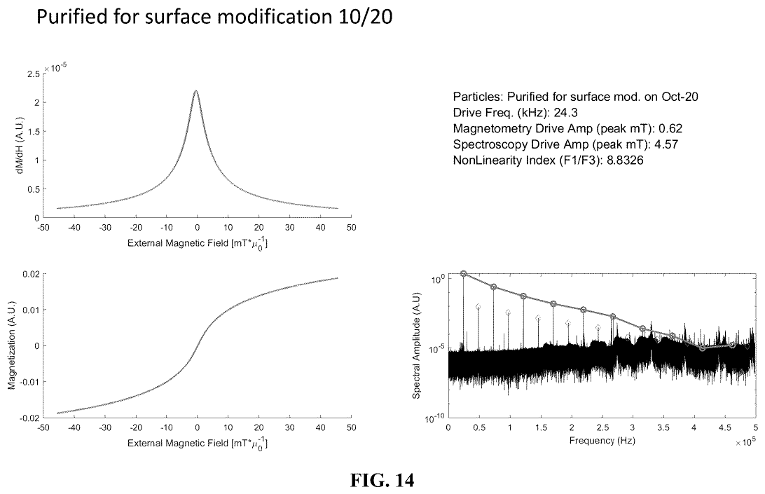

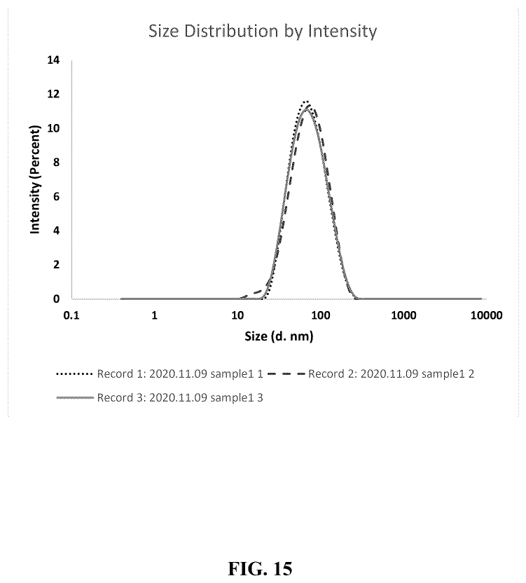

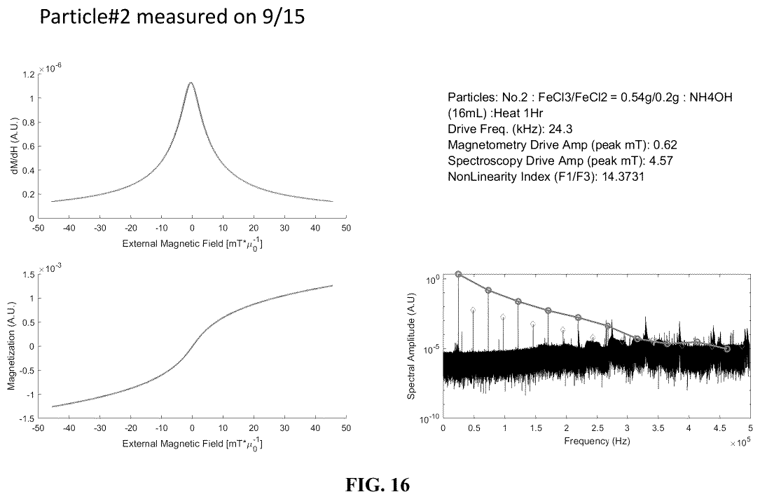

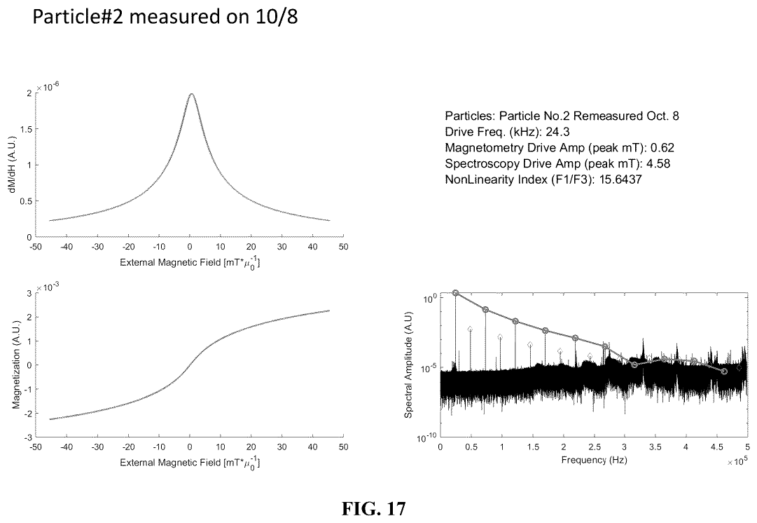

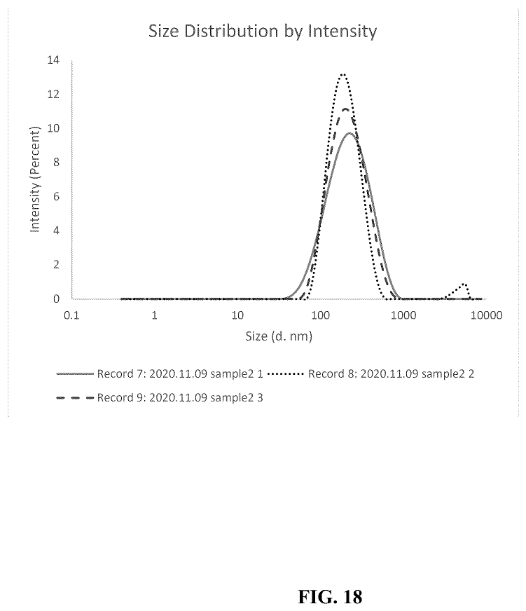

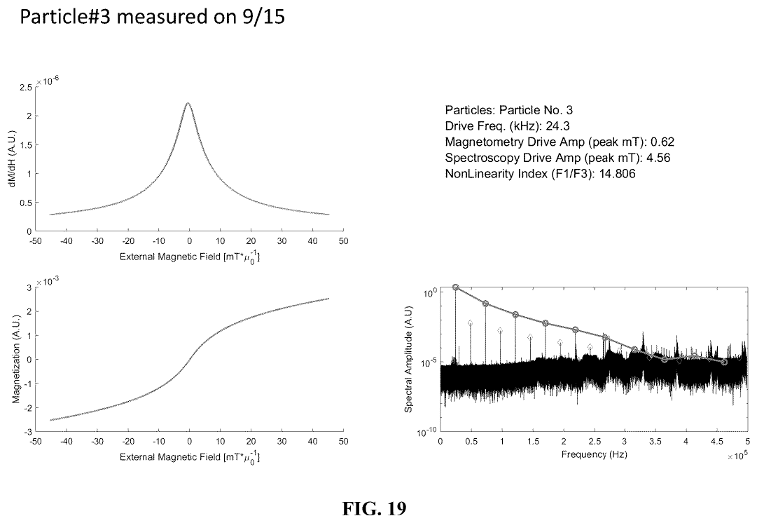

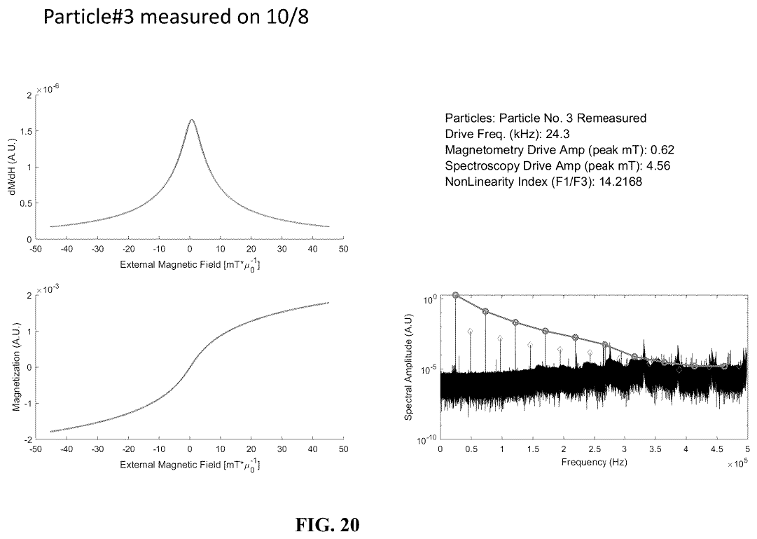

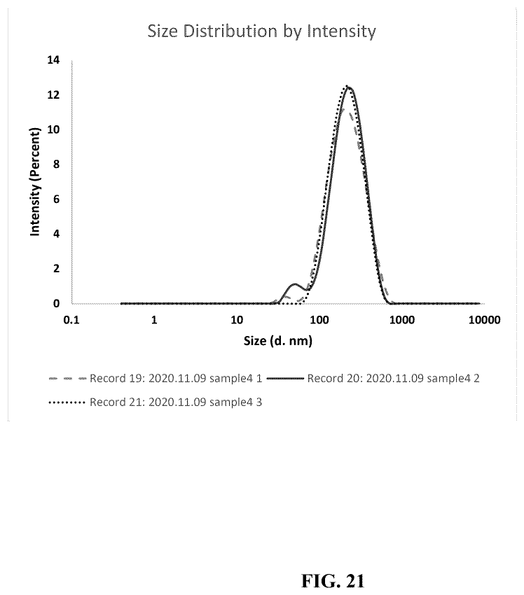

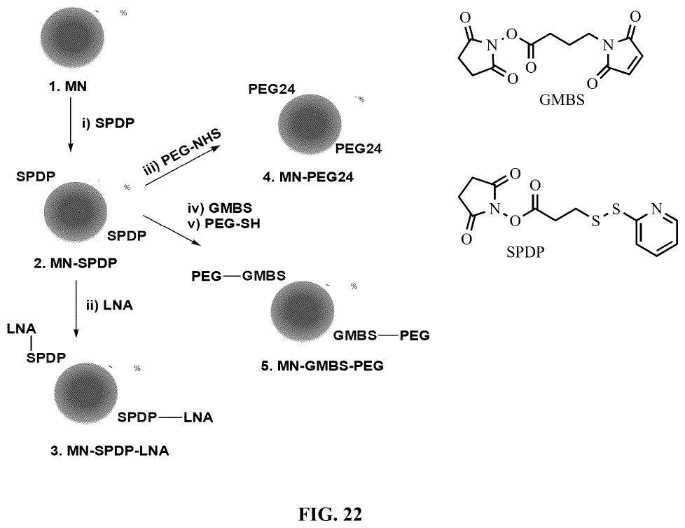

In some embodiments, the magnetic strength of the magnetic nanoparticles can be quantified by measuring a non-linearity index (NLI) by magnetic particle spectrometry. NLI is a criterion used to determine whether or not a particle is adequate for magnetic particle imaging or other techniques that rely on the non-linear behavior of magnetic nanoparticles. NLI can be determined by calculating a ratio of F1 to F3, which are parameters in the magnetic particle spectrometer system. F1/F3 compares the magnetization of particles versus an external magnetic field. F1 is the magnitude of an external magnetic excitation (“drive”) frequency following Fourier decomposition, and F3 refers to the magnitude of the third harmonic of the drive frequency (e.g. if the drive frequency is 25 kHz, F1 is 25 kHz and F3 is 75 kHz); thus, F1 and F3 are calculated with the magnitude of the frequency, and the process of Fourier decomposition makes it possible to analyze non-linear correlation in the time domain. If a particle has a magnetic property that is linearly proportional to the external magnetic field used by the magnetic particle spectrometer then its non-linearity index can be very large. If a particle has a magnetic property that is linearly proportional to the external magnetic field used by the magnetic particle spectrometer then its non-linearity index can be very large. The greater the magnetic permeability (“magnetic strength” or “dM/dH” in FIGS. 9, 11, 13, 14, 16, 17, 19, and 20) of a particle without an external magnetic field relative to the magnetic strength when magnetized by an external magnetic field, the smaller the non-linearity index will be (e.g., the closer it will approach 1, the NLI of a square wave magnetization response). Conversely, the more similar the initial magnetic strength of a particle relative to its fully magnetized state, the greater the non-linearity index will be. As NLI pertains to a specific excitation condition, the same external field has been used throughout all measurements present herein (a sinusoidal field with a peak magnitude of 4.5 mT/μ₀), though the methods and analysis can be similarly applied to other operating conditions.

In some embodiments, the nanoparticle compositions have an NLI ranging from about 6 to about 40. In some embodiments, the nanoparticle compositions have an NLI ranging from about 6 to about 70. In some embodiments, the nanoparticle compositions have an NLI ranging from about 8.5 to about 14.8. In some embodiments, the nanoparticle compositions have an NLI ranging from about 8 to about 14. In some embodiments, the nanoparticle compositions have an NLI of about 6. In some embodiments, the nanoparticle compositions have an NLI of about 8. In some embodiments, the nanoparticle compositions have an NLI of about 14. In some embodiments, the nanoparticle compositions have an NLI of about 67. In some embodiments, the nanoparticle compositions have an NLI ranging from 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 30, 30 to 40, 40 to 50, 50 to 60, or 60 to 70. In some embodiments, the nanoparticle compositions including about 0.54 g of ferric chloride and about 0.2 g of ferrous chloride have a non-linearity index ranging from about 8.5 to about 14.8. In some embodiments, the nanoparticle compositions including about 0.54 g of ferric chloride and about 0.2 g of ferrous chloride have a non-linearity index of about 12.

In some embodiments, the nanoparticle compositions include about 80% to about 100% of ferric chloride and about 20% to about 0% of ferrous chloride. In some embodiments, the nanoparticle compositions including about 0% to about 50% of ferric chloride and about 100% to about 50% of ferrous chloride have weaker magnetic properties than nanoparticle compositions having a ferrous chloride amount lower than about 0.4 g. In some embodiments, the nanoparticle compositions including about 0.54 g of ferric chloride and about 0.4 g of ferrous chloride have weaker magnetic properties than nanoparticle compositions having a ferrous chloride amount lower than about 0.2 g.

In some embodiments, the nanoparticle compositions including about 0.54 g of ferric chloride and about 0.4 g of ferrous chloride have a non-linearity index ranging from about 50 to about 120. In some embodiments, the nanoparticle compositions including about 0.54 g of ferric chloride and about 0.4 g of ferrous chloride have a non-linearity index of about 67.

Thus, in some embodiments, the magnetic properties (e.g., magnetic strength) of the magnetic nanoparticles can be fine-tuned by controlling the concentrations of ferric chloride and ferrous chloride, which are used to prepare the magnetic nanoparticles.

In some embodiments, the nanoparticle composition has an iron concentration ranging from about 8 μM to about 217 μM. In some embodiments, the nanoparticle composition has an iron concentration ranging from about 8 μM to about 15 μM, about 15 μM to about 25 μM, about 25 μM to about 50 μM, 50 μM to about 60 μM, about 60 μM to about 70 μM, about 70 μM to about 80 μM, 80 μM to about 90 μM, about 90 μM to about 100 μM, about 100 μM to about 110 μM, 110 μM to about 120 μM, about 120 μM to about 130 μM, about 130 μM to about 140 μM, 140 μM to about 150 μM, about 150 μM to about 160 μM, about 160 μM to about 170 μM, 170 μM to about 180 μM, about 180 μM to about 190 μM, about 190 μM to about 200 μM, 200 μM to about 210 μM, about 210 μM to about 220 μM.

In some embodiments, the nanoparticle composition has an iron concentration ranging from about 1 mg/mL to about 25 mg/mL. In some embodiments, the nanoparticle composition has an iron concentration ranging from about 1 mg/mL to about 5 mg/mL, about 5 mg/mL to about 10 mg/mL, about 10 mg/mL to about 15 mg/mL, about 15 mg/mL to about 20 mg/mL, or about 20 mg/mL to about 25 mg/mL.

### Other Physical Properties

In some embodiments, key properties of nanoparticles used for drug delivery include biodegradability, toxicity profile, and pharmacokinetics/pharmacodynamics of the nanoparticles. The composition and/or size of the nanoparticles are key determinants of their biological fate. For example, larger nanoparticles are typically taken up and degraded by the liver, whereas smaller nanoparticles (<30 nm in diameter) typically circulate for a long time (sometimes over 24-hr blood half-life in humans) and accumulate in lymph nodes and the interstitium of organs with hyperpermeable vasculature, such as tumors and metastases.

In some embodiments, the magnetic nanoparticles can have a diameter of between about 2 nanometers (nm) to about 200 nm (e.g., between about 2 nm to about 10 nm, between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm), e.g., at least about 2, 5, 10, 15, 20, 25, 50, 70, 80, 100, 120, 125, 140, or 150 nm, up to about 10, 20, 25, 30, 50, 75, 100, 150, 200, or 250 nm.

In some embodiments, the magnetic nanoparticles provided herein can be spherical or ellipsoidal or can have an amorphous shape. In some embodiments, the magnetic nanoparticles provided herein can have a diameter (between any two points on the exterior surface of the nanoparticle composition) of between about 2 nm to about 200 nm (e.g., between about 10 nm to about 200 nm, between about 2 nm to about 30 nm, between about 5 nm to about 25 nm, between about 10 nm to about 25 nm, between about 15 nm to about 25 nm, between about 20 nm to about 25 nm, between about 50 nm to about 200 nm, between about 70 nm to about 200 nm, between about 80 nm to about 200 nm, between about 100 nm to about 200 nm, between about 140 nm to about 200 nm, and between about 150 nm to about 200 nm). In some embodiments, magnetic nanoparticles having a diameter of between about 2 nm to about 30 nm localize to tumors, lymph nodes, and metastatic lesions in a subject. In some embodiments, magnetic nanoparticles having a diameter of between about 40 nm to about 200 nm localize to the liver.

In some embodiments, the magnetic nanoparticles provided herein can have a polydispersity index (PDI) of about 0.05 to about 0.25. The PDI is essentially a representation of the distribution of size populations within a given sample. The numerical value of PDI ranges from 0.0 (for a perfectly uniform sample with respect to the particle size) to 1.0 (for a highly polydisperse sample with multiple particle size populations). In some embodiments, the magnetic nanoparticles provided herein can have a PDI of about 0.050 to 0.100, about 0.100 to 0.110, about 0.110 to 0.120, about 0.120 to 0.130, about 0.130 to 0.140, about 0.140 to 0.150, about 0.150 to 0.160, about 0.160 to 0.170, about 0.170 to 0.180, about 0.180 to 0.190, about 0.190 to 0.200, about 0.200 to 0.210, about 0.210 to 0.220, about 0.230 to 0.240, or about 0.240 to 0.250.

In some embodiments, the magnetic material or particle can contain a diamagnetic, paramagnetic, superparamagnetic, or ferromagnetic material that is responsive to a magnetic field. Non-limiting examples of therapeutic magnetic nanoparticles contain a core of a magnetic material containing a metal oxide selected from the group of magnetite; ferrites (e.g., ferrites of manganese, cobalt, and nickel); Fe(II) oxides, and hematite, and metal alloys thereof. In some embodiments of the methods described herein, the position or localization of therapeutic magnetic nanoparticles can be imaged in a subject (e.g., imaged in a subject following the administration of one or more doses of a magnetic nanoparticle).

### Polymer Coatings

The magnetic nanoparticles described herein contain a polymer (e.g., dextran) coating over the core magnetic material (e.g., over the surface of a magnetic material). The polymer material can be suitable for attaching or coupling one or more biological agents (e.g., such as any of the nucleic acids described herein). One of more biological agents (e.g., a nucleic acid) can be attached to the polymer coating by chemical coupling (e.g., covalent bonds).

Method for the synthesis of iron oxide nanoparticles include, for example, physical and chemical methods. For example, iron oxides can be prepared by co-precipitation of Fe²⁺ and Fe³⁺ salts in an aqueous solution, e.g., as described in Examples 1-8. The resulting core consists of magnetite (Fe₃O₄), maghemite (γ-Fe₂O₃) or a mixture of the two. The anionic salt content (e.g., chlorides, nitrates, sulphates, etc.), the Fe²⁺ and Fe³⁺ ratio, pH, and the ionic strength in the aqueous solution all play a role in controlling the size of the nanoparticles. It is important to prevent the oxidation of the synthesized nanoparticles and protect their magnetic properties by carrying out the reaction in an oxygen-free environment under inert gas such as nitrogen or argon. The coating materials can be added during the co-precipitation process in order to prevent the agglomeration of the iron oxide nanoparticles into microparticles. Skilled practitioners will appreciate that any number of known surface coating materials can be used for stabilizing iron oxide nanoparticles, among which are synthetic and natural polymers, such as, for example, polyethylene glycol (PEG), dextran, polyvinylpyrrolidone (PVP), fatty acids, polypeptides, chitosan, gelatin. In some embodiments, the nanoparticle composition includes PEG. In some embodiments, the nanoparticle composition includes PEG-2000. In some embodiments, the nanoparticle composition includes PEG-1000, PEG-3000, PEG-3350, PEG-4000, PEG-6000, PEG-8000, PEG-12,000, PEG-20,000, or any combination thereof.

In some embodiments, the polymer coating is dextran. In some embodiments, the dextran coating is covalently linked to the magnetic nanoparticles. In some embodiments, the dextran coating includes dextran having a molecular weight ranging from about 1 kilodaltons (kDa) to about 15 kDa. In some embodiments, the dextran coating includes dextran having a molecular weight of about 1 kDa. In some embodiments, the dextran coating includes dextran having a molecular weight of about 5 kDa. In some embodiments, the dextran coating includes dextran having a molecular weight of about 10 kDa. In some embodiments, the dextran coating includes dextran having a molecular weight of about 15 kDa. In some embodiments, the dextran coating includes dextran that is chemically crosslinked, as described in Example 2. Alternative suitable polymers that can be used to coat the core of magnetic material include without limitation: polystyrenes, polyacrylamides, polyetherurethanes, polysulfones, fluorinated or chlorinated polymers such as polyvinyl chloride, polyethylenes, and polypropylenes, polycarbonates, and polyesters. Additional examples of polymers that can be used to coat the core of magnetic material include polyolefins, such as polybutadiene, polydichlorobutadiene, polyisoprene, polychloroprene, polyvinylidene halides, polyvinylidene carbonate, and polyfluorinated ethylenes. A number of copolymers, including styrene/butadiene, alpha-methyl styrene/dimethyl siloxane, or other polysiloxanes can also be used to coat the core of magnetic material (e.g., polydimethyl siloxane, polyphenylmethyl siloxane, and polytrifluoropropylmethyl siloxane). Additional polymers that can be used to coat the core of magnetic material include polyacrylonitriles or acrylonitrile-containing polymers, such as poly alpha-acrylanitrile copolymers, alkyd or terpenoid resins, and polyalkylene polysulfonates.

### Drug Payloads

In some embodiments, the nanoparticle compositions further include a drug payload. In some embodiments, the drug payload can be attached (e.g., via covalent bonding) to a surface of the dextran coating. In some embodiments, the drug payload is a drug, an antibody, a growth factor, a nucleic acid, a nucleic acid derivative, a nucleic acid fragment, a protein, a protein derivative, a protein fragment, a peptide, a small molecule, or any combination thereof. In some embodiments, the drug payload is an oligonucleotide conjugated to the one or more amine groups of the polymer coating (e.g., dextran coating). In some embodiments, the drug payload is a nucleic acid. In some embodiments, the nucleic acid is single-stranded or double-stranded. In some embodiments, the nucleic acid is an antisense RNA, a small interfering RNA (siRNA), a DNA, a microRNA mimic, an aptamer, or a ribozyme. In some embodiments, the nucleic acid molecule can contain at least one modified nucleotide (a nucleotide containing a modified base or sugar). In some embodiments, the nucleic acid molecule can contain at least one modification in the phosphate (phosphodiester) backbone. The introduction of these modifications can increase the stability or improve the hybridization or solubility of the nucleic acid molecule.

In some embodiments, the drug payload (e.g., a nucleic acid) is attached to the magnetic nanoparticle (e.g., to the polymer coating of the magnetic nanoparticle) through a chemical moiety that contains a thioether bond or a disulfide bond. In some embodiments, the nucleic acid is attached to the magnetic nanoparticle through a chemical moiety that contains an amide bond. Additional chemical moieties that can be used to covalently link a nucleic acid to the magnetic nanoparticle are known in the art.

A variety of different methods can be used to covalently link a drug payload to a magnetic nanoparticle. In some embodiments, carbodiimide is used for attachment of a drug payload to a magnetic nanoparticle.

### Pharmaceutical Compositions

Also provided herein are pharmaceutical compositions that include any of the nanoparticle compositions of the disclosure and at least one pharmaceutically acceptable carrier or diluent. In some embodiments, the pharmaceutical compositions include a magnetic nanoparticle as described herein. Two or more (e.g., two, three, or four) of any of the types of magnetic nanoparticles described herein can be present in a pharmaceutical composition in any combination. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfate, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the nanoparticle compositions can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid). Compositions containing one or more of any of the magnetic nanoparticles described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) magnetic nanoparticles (e.g., any of the magnetic nanoparticles described herein) will be an amount that decreases cancer cell invasion or metastasis in a subject having cancer, treats a metastatic cancer in a subject, decreases or stabilizes metastatic tumor size in in a subject, decreases the rate of metastatic tumor growth in a subject, decreases the severity, frequency, and/or duration of one or more symptoms of a metastatic cancer in a subject (e.g., a human), or decreases the number of symptoms of a metastatic cancer in a subject (e.g., as compared to a control subject having the same disease but not receiving treatment or a different treatment, or the same subject prior to treatment).

The effectiveness and dosing of any of the magnetic compositions described herein can be determined by a health care professional using methods known in the art, as well as by the observation of one or more symptoms of a metastatic cancer in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the nanoparticle compositions described herein per kilogram of the subject's weight. While these doses cover a broad range, one of ordinary skill in the art will understand that therapeutic agents, including the nanoparticle compositions described herein, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the nanoparticle compositions in vivo.

The pharmaceutical compositions can be included in a container, pack, or dispenser together with instructions for administration.

### Synthesis Methods

In some embodiments, provided herein are methods of preparing any of the nanoparticle compositions of the disclosure, as detailed in Examples 1-8. In some embodiments, the methods include dissolving dextran in water, preparing a ferrous chloride solution, a ferric chloride solution, or a combination thereof. In some embodiments, the methods include preparing a mixture by adding the ferrous chloride solution, the ferric chloride solution, or the combination thereof to the dextran.

In some embodiments, the methods include adding a base to the mixture while stirring and subjecting the mixture to an ice bath. In some embodiments, the methods include adding about 10 mL to 15 mL of a base to the mixture. In some embodiments, the methods include adding about 25 mL to 30 mL of a base to the mixture. In some embodiments, the methods include adding at least about 10 mL to 15 mL, 15 mL to 20 mL, 20 mL to 25 mL, 25 mL to 30 mL or more of a base to the mixture. In some embodiments, the base is ammonium hydroxide. In some embodiments, the base is sodium hydroxide. In some embodiments, the methods include adding about 10 mL of ammonium hydroxide to the mixture. In some embodiments, the methods include adding about 15 mL of ammonium hydroxide to the mixture. In some embodiments, the methods include adding about 25 mL of ammonium hydroxide to the mixture. In some embodiments, the methods include adding about 30 mL of ammonium hydroxide to the mixture.

In some embodiments, the methods include adding ammonium hydroxide to the mixture while stirring and subjecting the mixture to an ice bath. In some embodiments, an excess amount of ammonia or ammonium hydroxide is required to introduce an amine group at the same site of a hydroxyl group on the dextran coating. In some embodiments, the methods include adding about 60 mL of ammonium hydroxide to the mixture (e.g., the nanoparticle precursor composition). In some embodiments, the methods include subjecting the mixture to a temperature of about 75° C. to about 90° C. In some embodiments, the methods include subjecting the mixture to a temperature of about 75° C. to about 90° C. after ammonium hydroxide has been added. In some embodiments, the step of adding ammonium hydroxide prevents the formation of iron oxide crystals, iron oxide hydrates, or a combination thereof. In some embodiments, the step of adding ammonium hydroxide functionalizes the dextran coating with the one or more amine groups.

In some embodiments, the method includes crosslinking the dextran with epichlorohydrin. Epichlorohydrin is a chemical that can be used to crosslink two hydroxyl groups on the dextran polymer backbone. In some embodiments, the crosslinking by epichlorohydrin ensures the chemical stabilization of dextran coat on the surface of iron oxide core. In some embodiments, epichlorohydrin can polymerize to extend hydroxyl group chains on the dextran polymer backbone, which can result in the increase of hydroxyl groups that may be substituted with amine groups. In some embodiments, the addition of ammonium hydroxide to the mixture destroys the remained, unreacted epichlorohydrin in the reaction mixture.

In some embodiments, any of the nanoparticle compositions of the disclosure are amenable to be scaled up. For example, in some embodiments, the methods further include yielding a first final volume of a first nanoparticle composition of about 21 mL. In some embodiments, the first nanoparticle composition (e.g., a small-scale batch of magnetic nanoparticles) includes a first magnetic nanoparticle characterized by having a first set of physical properties. In some embodiments, the methods further include yielding a second final volume of a second nanoparticle composition (e.g., a large-scale batch of magnetic nanoparticles) at least greater than about 21 mL. In some embodiments, the second final volume of the second nanoparticle composition is about 20 mL to about 30 mL, about 30 mL to about 40 mL, about 40 mL to about 50 mL, about 50 mL to about 60 mL, about 60 mL to about 70 mL, about 70 mL to about 80 mL, about 80 mL to about 90 mL, about 90 mL to about 100 mL, about 100 mL to about 100 mL, or about 110 mL to about 120 mL.

In some embodiments, the second nanoparticle composition includes a second magnetic nanoparticle characterized by having a second set of physical properties. In some embodiments, the first and second set of physical properties are about the same. In some embodiments, any of the nanoparticle compositions of the disclosure can be scaled up without a change to its physical properties (e.g., size, PDI, or NLI). In some embodiments, any of the nanoparticle compositions of the disclosure can be scaled up without a change to its physical properties. In some embodiments, the first and second physical properties include a diameter, a magnetic strength, a polydispersity index, a surface charge, a non-linear index value, a PDI value, or any combination thereof.

In some embodiments, the nanoparticle compositions disclosed herein are stable for at least about 1 day to about 6 months or more. The term “stable” or “stability,” as used herein, indicates a lack of change in any of the physical properties of a same sample of the magnetic nanoparticles or compositions as measured and compared from the day when they were prepared to the day they are samples after being in storage. In some embodiments, the nanoparticle compositions disclosed herein are stable for at least about 1 day to about 5 days, for about 5 days to 10 days, about 10 days to about 15 days, for about 15 days to 30 days, about 30 days to about 40 days, for about 40 days to 50 days, about 50 days to about 60 days, about 3 months to about 4 months, about 4 months to about 5 months, about 5 months to about 6 months, or more.

### Methods of Treatment

In some embodiments, provided herein are methods of treating, preventing, or imaging a disease in a subject in need thereof. In some embodiments, the method includes administering a therapeutically effective amount of any of the nanoparticle compositions disclosed herein to at least a target site at a portion of a body, body part, tissue, cell, or body fluid of the subject. In some embodiments, any of the nanoparticle compositions of the disclosure are used in a method of treating a disease in a subject in need thereof. In some embodiments, any of the nanoparticle compositions of the disclosure are used in a method of imaging (e.g., via magnetic resonance imaging (MRI)) a disease in a subject in need thereof. In some embodiments, provided herein are methods of decreasing (e.g., a significant or observable decrease) cancer cell invasion or metastasis in a subject. In some embodiments, the methods include administering at least one nanoparticle composition described herein to the subject in an amount sufficient to decrease cancer cell invasion or metastasis in a subject.

In some embodiments, the methods further include administering energy to the magnetic nanoparticle composition and the target site. In some embodiments, the energy is light energy or magnetic energy. For example, in some embodiments, the step of administering energy can include administering a magnetic field or exposing a subject, which has been administered any of the nanoparticle compositions described herein, to a magnetic field for magnetic resonance imaging. In some embodiments, the nanoparticle compositions are used to image a portion of a body, body part, tissue, cell, or body fluid of the subject. In some embodiments, the nanoparticle compositions can treat, prevent (e.g., prevent further metastasis of a cancer cell by enabling detection of the cancer at an early stage), and/or image a disease. In some embodiments, the disease is cancer. In some embodiments, the disease is metastatic cancer. In some embodiments, the target site is a tumor site. In some embodiments, the nanoparticle composition accumulates at the target site of the subject (e.g., due to the size of the magnetic nanoparticles of the disclosure). In some embodiments, the methods further include imaging the target site using the nanoparticle composition. In some embodiments, the imaging is performed using magnetic resonance imaging.

In some embodiments, the step of administering energy to the magnetic nanoparticle composition and the target site is an optional step. For example, the magnetic compositions may be used as a therapeutic composition alone and not as both a therapeutic composition and an imaging agent (e.g., a contrast agent). In some embodiments, the magnetic compositions are used as an imaging agent (e.g., a contrast agent) alone and not as both a therapeutic composition and an imaging agent.

### Dosing, Administration, and Compositions

In any of the methods described herein, the nanoparticle compositions can be administered by a health care professional (e.g., a physician, a physician's assistant, a nurse, or a laboratory or clinic worker), the subject (i.e., self-administration). The administering can be performed in a clinical setting (e.g., at a clinic or a hospital), in an assisted living facility, or at a pharmacy.

In some embodiments of any of the methods described herein, the nanoparticle composition is administered to a subject that has been diagnosed as having a disease (e.g., cancer such as a primary cancer or a metastatic cancer). In some embodiments, the subject has been diagnosed with a metastatic cancer. Non-limiting examples of metastatic cancers include breast cancer, bladder cancer, colon cancer, kidney cancer, lung cancer, melanoma, ovarian cancer, pancreatic cancer, prostate cancer, rectal cancer, stomach cancer, thyroid cancer, and uterine cancer. In some non-limiting embodiments, the subject is a man or a woman, an adult, an adolescent, or a child. The subject can have experienced one or more symptoms of a cancer or metastatic cancer (e.g., a metastatic cancer in a lymph node). The subject can also be diagnosed as having a severe or an advanced stage of cancer (e.g., a primary or metastatic cancer). In some embodiments, the subject may have been identified as having a metastatic tumor present in at least one lymph node. In some embodiments, the subject may have already undergone lymphectomy and/or mastectomy.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the nanoparticle compositions or pharmaceutical compositions described herein. In any of the methods described herein, the at least one nanoparticle composition or pharmaceutical composition (e.g., any of the nanoparticle compositions or pharmaceutical compositions described herein) can be administered intravenously, intra-arterially, subcutaneously, intraperitoneally, or intramuscularly to the subject. In some embodiments, the at least magnetic particle or pharmaceutical composition is directly administered (injected) into a lymph node in a subject.

In some embodiments, the subject is administered at least one nanoparticle composition or pharmaceutical composition (e.g., any of the nanoparticle compositions or pharmaceutical compositions described herein) and at least one additional therapeutic agent. The at least one additional therapeutic agent can be a chemotherapeutic agent (e.g., cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, bortezomib, carfilzomib, salinosporamide A, all-trans retinoic acid, vinblastine, vincristine, vindesine, and vinorelbine) and/or an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, at least one additional therapeutic agent and at least one magnetic nanoparticle (e.g., any of the nanoparticle composition described herein) are administered in the same composition (e.g., the same pharmaceutical composition). In some embodiments, the at least one additional therapeutic agent and the at least one magnetic nanoparticle are administered to the subject using different routes of administration (e.g., at least one additional therapeutic agent delivered by oral administration and at least one magnetic nanoparticle delivered by intravenous administration).

In any of the methods described herein, the at least one nanoparticle composition or pharmaceutical composition (e.g., any of the nanoparticle compositions or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different nanoparticle compositions are administered in the same composition (e.g., a liquid composition). In some embodiments, at least one nanoparticle compositions and at least one additional therapeutic agent are administered in the same composition (e.g., a liquid composition). In some embodiments, the at least one nanoparticle compositions and the at least one additional therapeutic agent are administered in two different compositions (e.g., a liquid composition containing at least one nanoparticle compositions and a solid oral composition containing at least one additional therapeutic agent). In some embodiments, the at least one additional therapeutic agent is administered as a pill, tablet, or capsule.

In some embodiments, the at least one additional therapeutic agent is administered in a sustained-release oral formulation. In some embodiments, the one or more additional therapeutic agents can be administered to the subject prior to administering the at least one nanoparticle compositions or pharmaceutical composition (e.g., any of the nanoparticle compositions or pharmaceutical compositions described herein). In some embodiments, the one or more additional therapeutic agents can be administered to the subject after administering the at least one nanoparticle compositions or pharmaceutical composition (e.g., any of the magnetic particles or pharmaceutical compositions described herein). In some embodiments, the one or more additional therapeutic agents and the at least one nanoparticle compositions or pharmaceutical composition (e.g., any of the nanoparticle compositions or pharmaceutical compositions described herein) are administered to the subject such that there is an overlap in the bioactive period of the one or more additional therapeutic agents and the at least one nanoparticle compositions (e.g., any of the nanoparticle compositions described herein) in the subject.

In some embodiments, the subject can be administered the at least one nanoparticle composition or pharmaceutical composition (e.g., any of the nanoparticle compositions or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of nanoparticle compositions (and/or one or more additional therapeutic agents) administered to the subject and can also adjust (e.g., increase or decrease) the dosage or frequency of administration of at least one nanoparticle composition (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

### EXAMPLES

Certain embodiments of the present disclosure are further described in the following examples, which do not limit the scope of any embodiments described in the claims.

### Example 1—Synthesis of Magnetic Nanoparticles (MN) with a Modular Amino Payload

The synthesis of magnetic nanoparticles (MN) was carried out using an example set-up including a glass plate, with ice, containing a round-bottom flask. The round-bottom flask contained reaction components further described below. The round-bottom flask was placed on a hot plate/stir plate.

The formulation of the MN included dextran (9 g/30 mL D.I. water), 0.65 g Ferric chloride, 0.4 g Ferrous chloride, and 15 mL NH4OH (28%).

First, 9 grams of Dextran T10 was dissolved in deionized water (D.I. water) to make 30 mL (30% w/v) in a conical tube. Dextran T10 (technical quality) is a high purity dextran fraction with an average molecular weight of 10 kDa. A fresh solution of dextran was prepared as the solution forms precipitates within three days at room temperature.

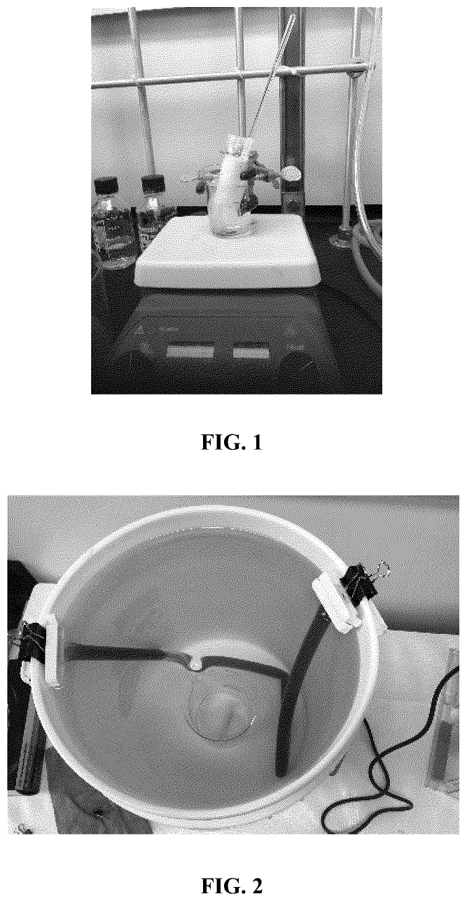

Next, dextran was solubilized in deionized (D.I.) water on a rotator at room temperature for 1 hr. The resulting solution was colorless, but it may look slightly cloudy with air bubbles. Moderate heat can be applied to dissolve the dextran completely. An example of the set-up for dextran dissolution is shown in FIG. 1.

The dextran solution was filtered using a 0.2 micrometer (μm)/0.45 μm filter into a 250 mL round bottom flask containing a magnetic stir bar. Any leftover dextran in the tube was may be with distilled water if necessary. The dissolved solution in the two-neck round bottom flask (Rbf) was chilled in an ice bath for 30 minutes with gentle magnetic stirring and nitrogen (or argon) bubbling (not air purging) to remove dissolved oxygen.

Next, the ferric chloride stock solution was prepared. The amount of ferric chloride used for “Condition 1” was 0.65 g of ferric chloride hexahydrate (FeCl₃·6H₂O), and 1.2 g of ferric chloride hexahydrate (FeCl₃·6H₂O) was used for “Condition 2.” The salts were dissolved in about 5 mL of DI water, as shown in Table 1. The stock solution exhibited a brown color, was filtered using a 0.22 μm filter unit, and was stored in a cold, dark place. The amount of iron was calculated by subtracting the other elements in the iron salt composition. The ferrous chloride tetrahydrate bottle was stored in a desiccator to minimize oxidation by air. The powder ferrous chloride should be a green color and formation of brown crystals in the bottle is an indication of iron oxidation (i.e., conversion from Fe(II) to Fe(III)), which should be avoided for obtaining high quality superparamagnetic nanoparticles.

Next, the ferrous chloride solution (FeCl₂·4H₂O) was prepared. 0.4 gr of ferrous chloride (Condition 1) were freshly weighed and dissolved in 1 mL of D.I. water within an Eppendorf tube resulting in a pearly light blue-green solution. 0.0 gr of ferrous chloride were used in the formulation of Condition 2. For the dissolution of ferrous chloride, D.I. water was purged with nitrogen for 10 minutes (min) to remove dissolved oxygen gas in water. Filtration was not needed after dissolution, but the dissolution step was carried out throughout 15 min (for 0.4 g of ferrous chloride—i.e., Condition 1) to make sure the complete dissolution was achieved. The amount of iron was calculated by ignoring the other elements in the iron salt composition.

0.65 g of ferric chloride in 1 mL (Condition 1), and 1.2 g of ferric chloride solution in 2-5 mL (Condition 2) of ferric chloride stock solution was added into the cold dextran solution. The mixture was stirred for an hour under a constant nitrogen (or argon) bubbling in the flask. After 30 min, 1 mL ferrous chloride solution (0.4 g FeCl₂ (condition 1) or (0.0 g FeCl₂ (condition 2) was added to the flask, as shown in Table 1. All necks of Rbf were tightly capped with a rubber stopper to prevent oxidation by minimizing air contact, but one neck had a gas outlet with a needle (18G) on top of rubber stopper.

TABLE 1
Formulations of Magnetic Nanoparticles (MN)
with a Modular Amino Payload

| | Condition | | Condition | |
|---|---|---|---|---|
| | 1 | | 2 | |
| FeCl₃•6H₂O (ferric chloride hexahydrate) | 0.65 | g | 1.2 | g |
| (FeCl₂•4H₂O) (ferrous chloride tetrahydrate) | 0.4 | g | 0 | g |
| Total iron salt added | 1.05 | g | 1.2 | g |
| Total Iron (Fe) added | 240 | mg | 240 | mg |

Next, the purging with inert gas was stopped. The cannular tube to add ammonium hydroxide without air contact was connected. At this step, the stirring speed was set maximum to overcome the changes in viscosity. The reaction mixture initially became very viscous and turned into an army-green color. Slow titration of ammonium hydroxide was performed. If ammonium hydroxide is added slowly, the viscosity increases to interfere the homogeneous mixing of ammonium hydroxide in ferric/ferrous mixture, resulting in large particles.

Vigorous stirring was continued in ice bath for 30 min. The ice bath under the reaction mixture was kept, and the stirring was maintained during the entire process. 60 minutes later, one neck was connected with a water-cooled condenser and the other neck was connected with the inert gas to purge (not in the reaction mixture) in high heat. Caution was used not to cause bumping in high temperature. The reaction Rbf was relocated into an oil bath, which was pre-heated to 90° C. Stirring was continued in the oil bath for 90 minutes. A thermometer was kept in the reaction mixture to measure temperature, and temperature was kept at about 75 to 85° C. at least. After this step the gas flow was stopped, and the solution was cooled to room temperature slowly. The formation of dextran coated magnetic nanoparticles was achieved at the end of these series of reactions. The volume of the final solution was around 40 mL.

The resulting solution was purified by Amicon tubes (50K centrifugal filter units) to remove unreacted dextran, iron salts, and ammonium hydroxide. The nanoparticle suspension was first concentrated with centrifugation (˜1,500×g (RCF) 3-4k RPM for 30 to 45 minutes), which resulted in a highly concentrated nanoparticle suspension on the filter and a nanoparticle-free elution under the filter unit. The eluent under the filter was discarded, and the nanoparticle pellet was re-suspended in D.I. water, and re-centrifuged using the same filter unit. This step was repeated until the eluent showed a pH of D.I. water or a neutral pH. Initially, centrifugation took about 1 hr due to the viscosity of the solution, large size of particle impurities, and the greater amount of unreacted, free dextran in the mixture. However, after the first 3 or 4 centrifugation steps, most of the free dextran was removed and re-suspension and concentration of nanoparticles was done in relatively short centrifugation steps (about 15 min per centrifugation step). The washing step was repeated 7 times. The resulting purified solution of magnetic nanoparticles was re-suspended in distilled water. The final volume was adjusted to 21 mL, and the solution was rested in a refrigerator (e.g., at about 4° C.) overnight.

### Example 2—Crosslinking and Amination

The nanoparticles were cross-linked and aminated with a series of reaction steps using sodium hydroxide, epichlorohydrin and ammonium hydroxide. 21 mL of MN were mixed with 35 mL sodium hydroxide (NaOH), 14 mL epichlorohydrin+60 mL ammonium hydroxide (NH4OH). The experiments were performed in a fume hood and safety precautions were taken in order to minimize exposure to the chemicals used in the synthesis. 35 mL of NaOH (5 M) was stored at 4° C. To prepare the 5M NaOH solution from pellets (ACROS 134070010, 1 kg, CAS 1310-732), 200 g were weighed and added to a glass bottle. 1 L water (milllipore) was added to bottle, the bottle was capped, and the mixture was swirled.

Cold 35 mL of NaOH (5 M) was added into the cold 21 mL of nanoparticle suspension in a 250 mL round bottom flask in an ice bath. The reaction mixture was stirred for 15 minutes without a gas flow in an ice bath. 14 mL of epichlorohydrin was added into the reaction mixture with vigorous stirring. The resulting solution formed two liquid phases after the addition of epichlorohydrin. After mixing, the temperature was maintained at room temperature. The cross-linking reaction continued for 8 hours with vigorous stirring at room temperature. The cross-linking reaction was exothermic, and the temperature was monitored and controlled so as to not exceed 35° C.

Epichlorohydrin was used to crosslink two hydroxyl groups on the dextran polymer backbone. The crosslinking by epichlorohydrin ensured the chemical stabilization of dextran coating on the surface of the iron oxide core of the MN. Epichlorohydrin is amenable to be polymerized in order to extend the chains, which can result in the increase of hydroxyl groups to be substituted with amine later.

The resulting homogenous solution was then reacted with ammonium hydroxide to aminate the final nanoparticle composition. 60 mL of ammonium hydroxide (NH₄OH, 28%) was added into the reaction mixture for both Condition 1 and Condition 2. The reaction mixture was stirred for 48 hours at room temperature. The neck of the round bottom flask was capped with a rubber stopper to prevent ammonia from evaporating, which is important for obtaining high yield of amination. After the reaction is over, the solution (˜150 mL) was transferred into a dialysis bag (MWCO 12-14 kDa) and dialyzed against 4-6 L of distilled water in a beaker with constant stirring in a fume hood. Dialysis was repeated several times over two days to remove all the unreacted ammonium hydroxide and side products (6-7 times). This was continued until the ammonia smell from the dialysis bag disappeared and the pH was neutral. After this, it was repeated 3-4 more times. An example of the dialysis set-up is shown in FIG. 2.

The resulting brownish black nanoparticle suspension was later concentrated to 20 mL using Amicon centrifuge units (MWCO 30 kDa, 2.8k rpm, 15 min.) The concentrated nanoparticles were suspended in 100 mM PBS buffer (pH 7.4). The solution was washed with PBS buffer one more time using Amicon centrifuge units (MWCO 30 kDa, 2.8k revolutions per minute (RPM), 15 min.) The volume was adjusted to 15 mL using PBS buffer (pH7.4). Nanoparticle solution was centrifuged at 14500 rpm. Afterwards, large particles were filtered off using 0.1 μm filter unit. An iron assay was performed to determine the amount of iron in solution. The volume was adjusted to make 12 mg Fe/mL using PBS buffer (pH=7.4). The size of the nanoparticle (about 22±3 nm in diameter) was determined by dynamic light scattering using Nanosizer.

### Example 3—Characterization of Iron Concentration of MN

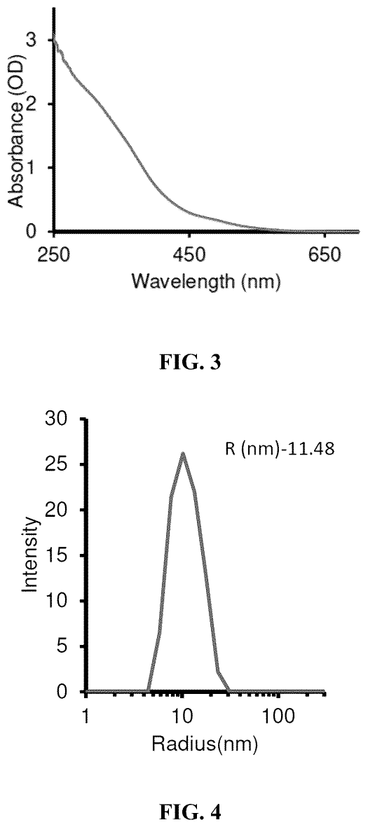

The iron content was determined by performing an iron assay as described below and utilized for the calculation of nanoparticle concentration. The amount of iron was determined using an iron assay described below using 8 standard iron solutions and 4 samples. 10 μL of iron standards and nanoparticle solution were added into 980 μL of 6 N HCl. 10 μL of hydrogen peroxide (H₂O₂, 30% in H₂O) were added into each mixture. A blank sample were prepared by adding 10 μL of distilled water instead of iron standards into the 980 μL of 6 N HCl and 10 μL of H₂O₂. The iron oxide cores were digested during this process. The optical density (OD) at 410 nm values was determined by UV-vis spectroscopy. The calibration curve was obtained using the standards. The concentration of the iron content in the nanoparticle solution was determined using the obtained calibration curve. An example of the UV-Vis curve is shown in FIG. 3. In prior experiments, the concentration was found to be between 8.7 μM (i.e., 1 mg/mL) of iron and 216.9 μM (i.e., 25 mg/mL) of iron.

### SPDP Quantification

725 μl water were added to 25 μl of conjugated nanoparticles. 2 tubes of the same dilution were prepared (i.e., with or without TCEP digestion). 25 μl of 3% TCEP were added. The solution was incubated at room temperature for about 10 minutes. Next, the solution was filtered through small Amicon filter (Eppendorf style; 100k cut off). The solution was then spun at 7000 RPM for about 2-5 min. The absorbance of the filtrate was measured at 343 nm. Absorbance data measured at 343 nm of filtrate with TCEP was about 0.33, and no peak was found in the filtrate without TCEP treatment. The total number of SPDP per nanoparticle was calculated as follows. Total no of SPDP: 0.33×10 6×30 (fold dil)/8100 (ext coefficient)=1200. Since nanoparticle concentration was 20 μM (2.2 mg/mL), the number of SPDP per nanoparticles was calculated by dividing 1200 SPDP by 10 μM and yielded 60 SPDP/μM NP.

### Example 4—Characterization of Nanoparticle Size and Amine Group Content

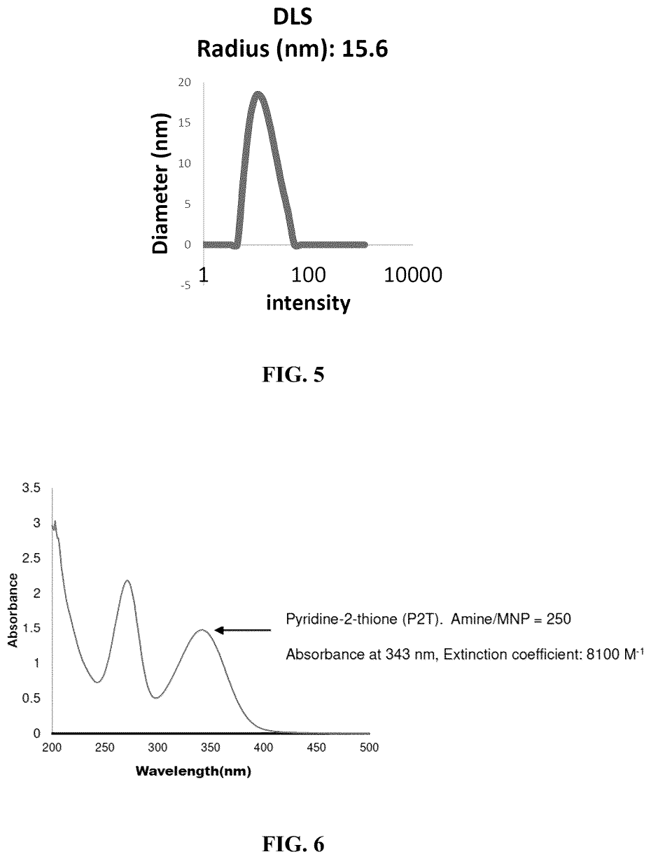

Nanoparticle size was determined using dynamic light scattering. In prior experiments, nanoparticles were synthesized with a radius as large as about 20 to 35 nm and as small as about 11.5 to 15.6 nm, as shown in FIGS. 4 and 5.

The amine content was quantified by the number of SPDPs (N-Succinimidyl 3-(2-pyridyldithio) propionate) that were conjugated to nanoparticles. SPDP is a hetero-bifunctional linker reactive to amino and sulfhydryl groups. SPDP-functionalized nanoparticles were cleaved by a reducing reagent (3% TCEP) to release a detectable by-product of pyridine-2-thione (P2T). Quantification of P2T was achieved by monitoring the maximum absorbance peak at 343 nm (extinction coefficient at 343 nm of 8.08×10³/cm/M). The number of P2T gives the number of reactive amine groups in the solution. The number of amine groups per nanoparticle was therefore, calculated by the ratio of concentration of P2T versus nanoparticles.

Briefly, an aliquot of nanoparticle suspension (100 μL) was diluted in 800 μL of Phosphate Buffered Saline (PBS, pH 7.4). The SPDP bottle was removed from freezer and equilibrated to room temperature before opening to avoid moisture accumulation in the bottle. This was important to prevent hydrolysis of the NHS ester of SPDP. A 100 mM SPDP stock solution was prepared in anhydrous DMSO. SPDP has limited water solubility therefore, the nanoparticle solution was titrated into the SPDP solution (in DMSO) slowly in order to prevent crystallization of SPDP. 100 μL of the nanoparticles were diluted with 800 μL of PBS buffer and 100 μL of 100 mM SPDP solution was added. The mixture was incubated on a rotator in a cold room (for about 16 to 20 hrs).

The nanoparticles were purified using disposable Sephadex PD-10 columns using PBS buffer as eluent. 1000 μL of eluent was collected. 450 μL of the purified SPDP-functionalized nanoparticles were mixed (out of ˜1000 μL after PD-10 column) with 50 μL of 3% TCEP, and the mixture was rested for 20 min at room temperature. TCEP reduces SPDP to release pyridine-2-thione, which is detectable by absorbance spectroscopy. Disulfide reducing agents, including DTT (dithiothreitol) or TCEP residues, or other contaminants were avoided in the mixture to maintain the activity of SPDP on the nanoparticle.

The reaction mixture was transferred into an Amicon filtration unit (0.5 mL, MWCO 100 kDa) and centrifuged in a microcentrifuge using ˜10,000×g (RCF) for 10 mins. The eluent, containing the P2T, was recovered and used for amine quantification by UV-vis spectroscopy. The retained nanoparticle pellet on filter unit was discarded. The amount of iron in the purified SPDP-functionalized nanoparticles solution was determined using an iron assay described below using 8 standard iron solutions and 4 samples.

Briefly, 10 μL of iron standards and nanoparticle solution were added into 980 μL of 6 N HCl. 10 μL of hydrogen peroxide (H₂O₂, 30% in H₂O) was added into each mixture. A blank sample was prepared by adding 10 μL of distilled water instead of iron standards into the 980 μL of 6 N HCl and 10 μL of H₂O₂. The iron oxide cores were digested during this process. The optical density (OD) at 410 nm values was determined by UV-vis spectroscopy. The calibration curve was obtained using the standards. The concentration of the iron content in the nanoparticle solution was determined using the obtained calibration curve.

The nanoparticle concentration was determined after measuring the iron concentration in the nanoparticle suspension by the assumption that each nanoparticle has an average of 2064 iron atoms per nanoparticle. In general, the concentration was determined to be about 12 mg/mL, which is equivalent to a 100 μM nanoparticle solution.

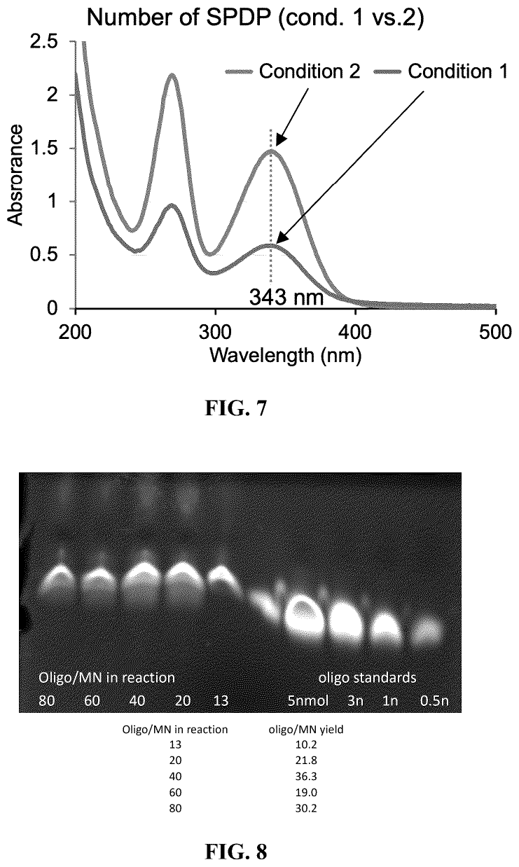

An unexpected and surprising result was found: by varying the amounts of FeCl₃ and FeCl₂ that were used in the reaction, the number of amino groups per nanoparticle was able to be modulated. Condition 1, including both FeCl₃ and FeCl₂ yielded about 60-90 amino groups per nanoparticle. Condition 2, resulted in the incorporation of about 246-500 amino groups per nanoparticle, which was an unusually high number. An example of the UV-Vis spectrum representing P2T absorbance at 410 nm is shown in FIGS. 6 and 7.

### Amine Group Quantification

100 μl of MN were mixed with 100 μl PBS in an Eppendorf tube and bring to 4° C. A a 20 mM solution of SPDP in DMSO (1 mg in 100 μl) was prepared. A cold nanoparticle solution was added to SPDP solution dropwise (reaction was exothermic). The solution was incubated at room temperature (RT) for 30 min. The solution was then purified through a PD-10 column and equilibrated with PBS using gravity. About 2 mL was collected. Two 350 μl aliquots (“sample” and “control”) were placed in two Amicon filter (microcons). 30 μl TCEP (35 mM) were added to the sample and the sample was left alone for 10 min. Both sample and control were spun down at 6000 RPM for 20 min at RT. 30 μl TCEP (35 mM) were added to control elute. Both sample and control were diluted at a ratio of 1:4.86 in PBS. The optical densities (OD) of the sample and control were read at 343 nm. The number of amine groups was calculated using the formulas shown below. In cuvette, sample was diluted 20*1.0857*4.86)=105.53 times. Concentration of iron in cuvette=Concentration of iron stock solution/105.53. [Crystals] in cuvette=[Fe] in cuvette/0.116 (constant)=[crystals] in μM. [Pyridine-2-thione] in cuvette=delta OD/0.0081 (ext coefficient)=[pyridine 2 thione] in μM NH2/xtal=[xtals] in cuvette/[pyridine-2-thione] in cuvette.

### Example 5—Conjugation of Oligonucleotides to MN

MN were functionalized with thiolated oligonucleotides, as described herein. A stock nanoparticle solution was prepared by mixing 10 mg Fe (equivalent to about 1 mL) in PBS buffer (pH 7.4). The nanoparticles were later conjugated to SPDP in order to provide thiol reactive terminals to nanoparticles for further conjugation steps. The SPDP bottle was removed from freezer and equilibrated to room temperature (for about 30 min) before opening the bottle to avoid moisture accumulation in the bottle, as indicated above. 10 mg of SPDP was dissolved in 500 μL of anhydrous DMSO, transferred into cold 13 mL Falcon tube and used immediately. The nanoparticle solution was titrated into the SPDP solution slowly via vortexing and pipetting. Fresh SPDP solution had to be prepared for each time since it hydrolyzes quickly.

After overnight incubation in the dark the nanoparticles are purified using disposable PD-10 column against PBS buffer (pH 7.4) to remove free unreacted SPDP molecules. Discard the last part of nanoparticles band in the column to separate free SPDP from nanoparticles completely. The concentration of final nanoparticle solution was calculated using iron assay. The nanoparticles with thiol reactive ends were then conjugated to the thiol-modified oligonucleotides. The thiol-modified oligonucleotides were dissolved in nuclease free water to a final concentration of 1 mM. The oligonucleotides were then treated with 3% tris(2-carboxyethyl)phosphine (TCEP) in order to activate the thiol groups by cleaving the protecting disulfide bonds in the oligonucleotide construct. The 3% TCEP was prepared freshly before each use. 100 μL of TCEP solution was added to the 1000 μL of oligonucleotide stock solution (1 mM) and incubated for 10 minutes. Later the oligonucleotides were purified using ammonium acetate/ethanol precipitation method.

Briefly, 500 μL of 9.5 M ammonium acetate was added to the oligonucleotide mixture. Later, 2300 μL of cold ethanol (200 proof, molecular biology grade) was added to the mixture. The white cloudy oligonucleotide precipitation was observed in the tube. The solution was then left at −80° C. for one hour. Later, the oligonucleotide mixture was centrifuged at 4° C. for fifteen minutes at 20,000×g (RCF). A white oligonucleotide pellet formed at the bottom of the tube after the end of the centrifugation. The supernatant was discarded, and the pellet was washed several times with 100% ethanol and 70% ethanol in water. The pellet was later dried by speed vacuum concentrator and re-suspended in nuclease free water to a final concentration of 1 mM. The nanoparticles were mixed with activated oligonucleotides with a 1 to 13 (up to 1:40) molar ratio on a rotator in the cold room at least one day. The nanoparticle solution was filtered with a 0.22 μm syringe filter to remove any large contaminants. For in vitro or in vivo studies, 100 μL of nanoparticles were purified using a G-50 Sephadex disposable quick spin columns in PBS (pH 7.4).

The concentration, size, and oligonucleotide loading of the resulting therapeutic iron oxide nanoprobes were characterized using iron assay, dynamic light scattering, and gel electrophoresis. The nanoparticles were concentrated using 0.5 mL amicon filtration units (MWCO 100 kDa, Amicon Ultra-0.5 mL Centrifugal Filters) with centrifugation if necessary for in vivo studies with small animals. An example of gel electrophoresis for the analysis of oligonucleotide loading is shown in FIG. 8. By varying the ratio of oligo to amino groups/nanoparticle, the number of oligos/nanoparticle can be progressively increased and fine-tuned.

### Analysis of Oligo Loading in Polyacrylamide Gels

An appropriate quantity (e.g., 10 μl) of TCEP-digested MN was added to an Eppendorf tube. Free oligo was used as control to locate band in gel and quantify. 2 μl of nucleic acid loading buffer (5×) were added and mixed. Each sample was heated at 70° C. for 3 min. Each sample was cooled to RT and spun it down quickly. The entire liquid was loaded carefully on a 15% TBE-urea (polyacrylamide) gel or 4-20% PAGE. The gel was run using 1×TBE buffer for about 30-40 min at 130 volts. The gel was removed from the plastic cassette carefully. The gel was stained with ethidium bromide (1 μg/mL; 5 μl stock added to 50 mL water) for 20 min. The ethidium bromide solution was decanted and saved to properly dispose of it later and the gel was washed twice with water for about 5 min. each time. Next, the gels were visualized under UV light.

### Example 6—Synthesis of Magnetic Nanoparticles (MN) with Controllable Magnetic Properties

The synthesis of magnetic nanoparticles (MN) was carried out using an example set-up including a glass plate, with ice, containing a round-bottom flask. The round-bottom flask contained reaction components further described below. The round-bottom flask was placed on a hot plate/stir plate.

The formulation of the MN included dextran (9 g/30 mL D.I. water), 0.54 g Ferric chloride, 0.24 g Ferrous chloride, and 1 mL NH4OH (28%). This formulation yielded minimal non-linearity index in magnetic property.

First, 9 grams of Dextran T10 was dissolved in deionized water (D.I. water) to make 30 mL (30% w/v) in a conical tube. Dextran T10 (technical quality) is a high purity dextran fraction with an average molecular weight of 10 kDa. A fresh solution of dextran was prepared as the solution forms precipitates within three days at room temperature.

Next, dextran was solubilized in deionized (D.I.) water on a rotator at room temperature for 1 hr. The resulting solution was colorless, but it may look slightly cloudy with air bubbles. Moderate heat can be applied to dissolve the dextran completely. An example of the set-up for dextran dissolution is shown in FIG. 1.

The dextran solution was filtered using a 0.2 micrometer (μm)/0.45 μm filter into a 250 mL round bottom flask containing a magnetic stir bar. Any leftover dextran in the tube was may be with distilled water if necessary. The dissolved solution in the two-neck round bottom flask (Rbf) was chilled in an ice bath for 30 minutes with gentle magnetic stirring and nitrogen (or argon) bubbling (not air purging) to remove dissolved oxygen.

Next, the ferric chloride stock solution was prepared. The amounts of ferric chloride and ferrous chloride were 0.54 g of ferric chloride hexahydrate (FeCl₃·6H₂O) and 0.2 g of ferrous chloride tetrahydrate (FeCl₂·4H₂O) for “Condition 1.” and 0.54 g of ferric chloride hexahydrate (FeCl₃·6H₂O) and 0.4 g of ferrous chloride tetrahydrate (FeCl₂·4H₂O) for “Condition 2.”. The salts were dissolved in about 5 mL of DI water, as shown in Table 2. The stock solution exhibited a brown color, was filtered using a 0.22 μm filter unit, and was stored in a cold, dark place. The amount of iron was calculated by subtracting the other elements in the iron salt composition. The ferrous chloride tetrahydrate bottle was stored in a desiccator to minimize oxidation by air. The powder ferrous chloride should be a green color and formation of brown crystals in the bottle is an indication of iron oxidation (i.e., conversion from Fe(II) to Fe(III)), which should be avoided for obtaining high quality superparamagnetic nanoparticles.

Next, the ferrous chloride solution (FeCl₂·4H₂O) was prepared. 0.2 g of ferrous chloride (Condition 1) were freshly weighed and dissolved in 1 mL of D.I. water within an Eppendorf tube resulting in a pearly light blue-green solution. 0.4 gr of ferrous chloride was used in the formulation of Condition 2. For the dissolution of ferrous chloride, D.I. water was purged with nitrogen for 10 minutes (min) to remove dissolved oxygen gas in water that can produce non-magnetic oxidized iron (rust). Filtration was not needed after dissolution, but the dissolution step was carried out throughout 15 min (for 0.4 g of ferrous chloride—i.e., Condition 1) to make sure the complete dissolution was achieved. The amount of iron was calculated by ignoring the other elements in the iron salt composition.

0.545 g of ferric chloride in 1 mL of ferric chloride stock solution (Condition 1 and Condition 2) was added into the cold dextran solution. 1 mL ferrous chloride solution (0.2 g FeCl₂ (condition 1) or (0.4 g FeCl₂ (condition 2)) was added to the flask, as shown in Table 2. The mixture was stirred for an hour under a constant nitrogen (or argon) bubbling in the flask. All necks of Rbf were tightly capped with a rubber stopper to prevent oxidation by minimizing air contact, but one neck had a gas outlet with a needle (18G) on top of rubber stopper.

TABLE 2
Formulations of Magnetic Nanoparticles (MN) with Controllable
Magnetic Properties

| | Condition 1 | | Condition 2 | |
|---|---|---|---|---|
| FeCl₃•6H₂O (ferric chloride hexahydrate) | 0.54 | g | 0.54 | g |
| (FeCl₂•4H₂O) (ferrous chloride tetrahydrate) | 0.2 | g | 0.4 | g |
| Total iron salt added | 0.74 | g | 0.94 | g |
| Total Iron (Fe) added | 168 | mg | 224 | mg |

1. Next, the purging with inert gas was stopped. The cannular tube to add ammonium hydroxide without air contact was connected. 1 mL of concentrated cold (˜4° C.) ammonium hydroxide (NH₄OH, 280) was quickly added into the reaction mixture in ice bath. At this step, the stirring speed was set maximum to overcome the changes in viscosity. If ammonium hydroxide is added slowly, the viscosity increases to interfere the homogeneous mixing of ammonium hydroxide in ferric/ferrous mixture, resulting in large particles. It was ensured that extra ammonium hydroxide or less than 1 mL of ammonium hydroxide was not added.

TABLE 3

| Dextran | FeCl₃ | FeCl₃ | | | Heating | | Non-Linearity Index |
|---|---|---|---|---|---|---|---|
| (30%) | (0.54 g) | (0.2 g) | NH₄OH | | Time | | (NLI) |
| 30 mL | 1 eq | 1 eq | 0.6 | mL | 1 | hr | precipitates |
| | | | 1 | mL | 1 | hr | 9.5589 |
| | | | 2 | mL | 1 | hr | 65.0806 |
| | | | 3 | mL | 1 | hr | 62.9234 |
| | | | 4 | mL | 1 | hr | 113.1649 |
| 30 mL | 1 eq | 1 eq | 0.6 | mL | 2 | hr | precipitates |
| | | | 1 | mL | 2 | hr | 14.2824 |
| | | | 2 | mL | 2 | hr | 100.5543 |
| | | | 3 | mL | 2 | hr | 238.4305 |
| | | | 4 | mL | 2 | hr | 453.4567 |

Vigorous stirring was continued in ice bath for 15 min. The ice bath under the reaction mixture was kept, and the stirring was maintained during the entire process. 15 minutes later, one neck was connected with a water-cooled condenser and the other neck was connected with the inert gas to purge (not in the reaction mixture) in high heat. Caution was used not to cause bumping in high temperature. The reaction Rbf was relocated into an oil bath, which was pre-heated to 90° C. Stirring was continued in the oil bath for 60 minutes. A thermometer was kept in the reaction mixture to measure temperature, and temperature was kept at about 75 to 85° C. at least. The mixture was not heated for more than 60 minutes. After this step the gas flow was stopped, and the solution was cooled to room temperature slowly. The formation of dextran coated magnetic nanoparticles was achieved at the end of these series of reactions. The volume of the final solution was less than 40 mL. Stirring was continued at room temperature for 12 hours. The volume was set to 40 mL by adding D.I. water. The solution was transferred into a 50 mL conical tube and large particles were removed by centrifugation at 14,000 RPM for 1 hr. The solution was transferred into Amicon filter units (10 mL×4), and the particles were discarded in a 50 mL conical tube.

The resulting solution was purified by Amicon tubes (50K centrifugal filter units) to remove unreacted dextran, iron salts, and ammonium hydroxide. The nanoparticle suspension was first concentrated with centrifugation (4,500 RPM for 3 hours), which resulted in a highly concentrated nanoparticle suspension on the filter and a nanoparticle-free elution under the filter unit. The eluent under the filter was discarded, and the nanoparticle gel-like pellet was re-suspended in D.I. water, and re-centrifuged using the same filter unit. This step was repeated until the eluent showed a pH of D.I. water or a neutral pH. Initially, centrifugation took about 1 hr due to the viscosity of the solution, large size of particles, and the greater amount of unreacted, free dextran in the mixture. However, after the first 3 or 4 centrifugation steps, most of the free dextran was removed and re-suspension and concentration of nanoparticles was done in relatively short centrifugation steps (about 15 min per centrifugation step). The washing step was repeated 7 times. The resulting purified solution of magnetic nanoparticles was re-suspended in distilled water. The final volume was adjusted to 21 mL, and the solution was rested in a refrigerator (e.g., at about 4° C.) overnight.

### Example 7—Characterization of Magnetic Properties of MN

The samples were analyzed by magnetic particle spectrometer (MPS), and the non-linearity index (NLI) was used as a criterion for magnetic property of nanoparticles. FIGS. 9, 11, 13-14, 16-17, and 19-20 show example MPS analysis data including NLI values for each MN sample. The surface modification steps for the synthesis and characterization of these MN were the same as described in the previous Examples.

Magnetic properties were controlled by modulating the ratio of ferrous chloride and ferric chloride in the reaction mixture. To improve the suspension stability of nanoparticles in aqueous media, the control of magnetic property is critical. In this system, the surface was designed to equip the surface with positive charges, which can overcome the magnetic attraction between particles in Brownian motion that could result in the coagulation/instability of nanoparticles during long-term storage. The degree of amination per particle was larger than 64, which ensured the suspension stability in aqueous media.

In terms of magnetic properties, non-linearity index (NLI) is a well characterized property of magnetic particles used to quantify the responsiveness to an external magnetic field. When the particles have stronger magnetic properties (permeability) without an external magnetic field relative to the properties with a given magnetic field applied, NLI becomes smaller and the relationship shows non-linear correlation to external magnetic field, and thus is more well-suited for imaging and therapeutic techniques that rely on said nonlinearity, one example being magnetic particle imaging (MPI).

### Example 8—Synthesis of MN at Different Scales

The synthesis of magnetic nanoparticles (MN) was carried out using an example set-up including a glass plate, with ice, containing a round-bottom flask. The round-bottom flask contained reaction components further described below. The round-bottom flask was placed on a hot plate/stir plate.

The formulation of the MN included dextran (18 g/60 mL D.I. water), 0.54 g Ferric chloride, 0.2 g Ferrous chloride, and 1 mL NH4OH (28%). This formulation yielded minimal non-linearity index in magnetic property.

First, 18 grams of Dextran T10 was dissolved in deionized water (D.I. water) to make 60 mL (30% w/v) in a conical tube. Dextran T10 (technical quality) is a high purity dextran fraction with an average molecular weight of 10 kDa. A fresh solution of dextran was prepared as the solution forms precipitates within three days at room temperature.

Next, dextran was solubilized in deionized (D.I.) water on a rotator at room temperature for 1 hr. The resulting solution was colorless, but it may look slightly cloudy with air bubbles. Moderate heat can be applied to dissolve the dextran completely. An example of the set-up for dextran dissolution is shown in FIG. 1.

The dextran solution was filtered using a 0.2 micrometer (μm)/0.45 μm filter into a 250 mL round bottom flask containing a magnetic stir bar. Any leftover dextran in the tube was may be with distilled water if necessary. The dissolved solution in the two-neck round bottom flask (Rbf) was chilled in an ice bath for 30 minutes with gentle magnetic stirring and nitrogen (or argon) bubbling (not air purging) to remove dissolved oxygen.

Next, the ferric chloride stock solution was prepared. The amount of ferric chloride was 0.54 g of ferric chloride hexahydrate in 100 mL of DI water, as shown in Table 4 below. The stock solution exhibited a brown color, was filtered using a 0.22 μm filter unit, and was stored in a cold, dark place. The amount of iron was calculated by subtracting the other elements in the iron salt composition. The ferrous chloride tetrahydrate bottle was stored in a desiccator to minimize oxidation by air. The powder ferrous chloride should be a green color and formation of brown crystals in the bottle is an indication of iron oxidation (i.e., conversion from Fe(II) to Fe(III)), which should be avoided for obtaining high quality superparamagnetic nanoparticles.

Next, the ferrous chloride solution (FeCl₂·4H₂O) was prepared. 0.20 gr of ferrous chloride (Condition 1) were freshly weighed and dissolved in 1 mL of D.I. water within an Eppendorf tube resulting in a pearly light blue-green solution. 0.4 gr of ferrous chloride were used in the formulation of Condition 2. For the dissolution of ferrous chloride, D.I. water was purged with nitrogen for 10 minutes (min) to remove dissolved oxygen gas in water. Filtration was not needed after dissolution, but the dissolution step was carried out throughout 10 min (for 0.4 g of ferrous chloride—i.e., Condition 1) to make sure the complete dissolution was achieved.

Ferric chloride stock solution was added into the cold dextran solution. 1 mL ferrous chloride solution 1 eq. 0.2 g FeCl₂ was added to the flask, as shown in Table 4. The mixture was stirred for an hour under a constant nitrogen (or argon) bubbling in the flask. All necks of Rbf were tightly capped with a rubber stopper to prevent oxidation by minimizing air contact, but one neck had a gas outlet with a needle (18G) on top of rubber stopper.

TABLE 4
Formulations of Magnetic Nanoparticles (MN) for Scale-Up

| | Dextran | FeCl₃ | | FeCl₂ | | | | Heating | | |
|---|---|---|---|---|---|---|---|---|---|---|
| Condition | (30%) | (0.54 g) | | (0.2 g) | | NH4OH | | Time | NLI | |
| A | 30 mL | 1 | eq | 1 | eq | 1 mL | (0.5 mL × 2) | 1 hr | 9.5589 | |
| B | 30 mL | 6 | eq | 6 | eq | 6 mL | (1 mL × 6) | 1 hr | 9.7111 | |
| C | | 6 | eq | 6 | eq | 6 mL | (1 mL × 6) | 1 hr | 8.4556 | NLI = 8.8326(after |
| | | | | | | | | | (before | purification) |
| | | | | | | | | | purified) | |
| D | 60 mL | 6 | eq | 6 | eq | 8 mL | (2 mL × 3) | 1 hr | 14.2014 | |
| E | | 12 | eq | 12 | eq | 16 mL | (4 mL × 3) | 1 hr | 14.3731 | NLI = 15.6437 |
| | | | | | | | | | | (1 month, 5° C.) |
| F | | 18 | eq | 18 | eq | 24 mL | (6 mL × 3) | 1 hr | 14.806 | NLI = 14.2168 |
| | | | | | | | | | | (1 month, 5° C.) |

Next, the purging with inert gas was stopped. The cannular tube to add ammonium hydroxide without air contact was connected. 1 mL of concentrated cold (˜4° C.) ammonium hydroxide (NH₄OH, 28%) was quickly added into the reaction mixture in ice bath. At this step, the stirring speed was set maximum to overcome the changes in viscosity. The reaction mixture initially became very viscous and turned into an army-green color. The viscosity was lost after the ammonium hydroxide titration was over. If ammonium hydroxide is added slowly, the viscosity increases to interfere the homogeneous mixing of ammonium hydroxide in ferric/ferrous mixture, resulting in large particles. It was ensured that extra ammonium hydroxide or less than 1 mL of ammonium hydroxide was not added.

Vigorous stirring was continued in ice bath for 15 min. The ice bath under the reaction mixture was kept, and the stirring was maintained during the entire process. 15 minutes later, one neck was connected with a water-cooled condenser and the other neck was connected with the inert gas to purge (not in the reaction mixture) in high heat. Caution was used not to cause bumping in high temperature. The reaction Rbf was relocated into an oil bath, which was pre-heated to 60° C. Stirring was continued in the oil bath for 90 minutes. A thermometer was kept in the reaction mixture to measure temperature, and temperature was kept at about 75 to 85° C. at least. The mixture was not heated for more than 60 minutes. After this step the gas flow was stopped, and the solution was cooled to room temperature slowly. The formation of dextran coated magnetic nanoparticles was achieved at the end of these series of reactions. The volume of the final solution was around 40 mL. Stirring was continued at room temperature for 12 hours. The volume was set to 40 mL by adding D.I. water. The solution was transferred into a 50 mL conical tube and large particles were removed by centrifugation at 14,000 RPM for 1 hr. The solution was transferred into Amicon filter units (10 mL×4), and the particles were discarded in a 50 mL conical tube.

The resulting solution was purified by Amicon tubes (50K centrifugal filter units) to remove unreacted dextran, iron salts, and ammonium hydroxide. The nanoparticle suspension was first concentrated with centrifugation (4,500 RPM for 3 hours), which resulted in a highly concentrated nanoparticle suspension on the filter and a nanoparticle-free elution under the filter unit. The eluent under the filter was discarded, and the nanoparticle pellet was re-suspended in D.I. water, and re-centrifuged using the same filter unit. This step was repeated until the eluent showed a pH of D.I. water or a neutral pH. Initially, centrifugation took about 3 hr due to the viscosity of the solution, large size of particles, and the greater amount of unreacted, free dextran in the mixture. However, after the first 3 or 4 centrifugation steps, most of the free dextran was removed and re-suspension and concentration of nanoparticles was done in relatively short centrifugation steps (about 15 min per centrifugation step). The washing step was repeated 7 times. The resulting purified solution of magnetic nanoparticles was re-suspended in distilled water. The final volume was adjusted to 21 mL, and the solution was rested in a refrigerator (e.g., at about 4° C.) overnight. The samples were analyzed by magnetic particle spectrometer (MPS), and the non-linearity index (NLI) was calculated. The NLI values were used as a criterion for magnetic property of nanoparticles.

This scale-up study demonstrated a scale-up of 18 times larger than the studies described in Examples 1-6 in terms of total iron concentration. The main huddle that was overcome was the high viscosity in the step of iron oxide crystal formation, the step of ammonium hydroxide addition. The use of mechanical stirrer solved the issue of homogeneous mixing in the step described above and the addition of ammonium hydroxide was performed in the shortest time possible by pouring a pre-fixed volume, and with no titration. The volume of ammonium hydroxide is proportional to the amount of total iron compounds as shown in Table 4. The increase of dextran solution versus total iron concentration decreased the viscosity in the crystal formation step. These results demonstrated the mass production of magnetic nanoparticles with excellent non-linearity index in harsh condition of 18 eq total iron concentration in 60 mL dextran solution.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-31. (canceled)

32. A pharmaceutical composition comprising:
a magnetic nanoparticle comprising an iron oxide core;
a dextran coating functionalized with a number of surface amine groups from about 30 to about 150;
one or more oligonucleotides covalently attached to the surface amine groups; and
at least one pharmaceutically acceptable carrier or diluent,
wherein the composition comprises an iron concentration of about 1 mg/ml to about 15 mg/ml and has a polydispersity index (PDI) of about 0.050 to 0.100.

33. The pharmaceutical composition of claim 32, wherein the surface amine groups are present from about 40 to 110.

34. The pharmaceutical composition of claim 32, wherein the surface amine groups are present from about 75 to 110.

35. The pharmaceutical composition of claim 32, wherein the one or more oligonucleotides comprise one or more modified DNA nucleotides and/or one or more modifications in the phosphodiester bond linking two nucleotides.

36. The pharmaceutical composition of claim 35, wherein the one or more modified DNA nucleotides comprise one or more locked nucleotides.

37. The pharmaceutical composition of claim 32, wherein the one or more oligonucleotides are single stranded antisense oligonucleotides.

38. The pharmaceutical composition of claim 32, wherein the one or more oligonucleotides are covalently attached to the amine groups via a cleavable linker.

39. The pharmaceutical composition of claim 38, wherein the cleavable linker is a glutathione cleavable linker.

40.-42. (canceled)

43. The pharmaceutical composition of claim 32, configured for sustained delivery of the one or more oligonucleotides to a tumor.

44. The pharmaceutical composition of claim 32, formulated in a unit dosage for intravenous, intraarterial, intramuscular, intradermal, subcutaneous, or intraperitoneal administration.
